SEQUENCE LISTING (48 sequences)

SEQ ID NO: 1 - protein, 864 aa, Homo sapiens
MRMCTPIRGLLMALAVMFGTAMAFAPIPRITWEHREVHLVQFHEPDIYNYSALLLSEDKDTLYIGAREAVFAVNALNISEKQHEVYWKVSEDKKAKCAEKGKSKQTECLNYIRVLQPLSATSLYVCGTNAFQPACDHLNLTSFKFLGKNEDGKGRCPFDPAHSYTSVMVDGELYSGTSYNFLGSEPIISRNSSHSPLRTEYAIPWLNEPSFVFADVIRKSPDSPDGEDDRVYFFFTEVSVEYEFVFRVLIPRIARVCKGDQGGLRTLQKKWTSFLKARLICSRPDSGLVFNVLRDVFVLRSPGLKVPVFYALFTPQLNNVGLSAVCAYNLSTAEEVFSHGKYMQSTTVEQSHHRKWVRYNGPVPKPRPGLACIDSEARAANYTSSLNLPDKTLQFVKDHPLMDDSVTPIDNRPRLIKKDVNYTQIVVDRTQALDGTVYDVMFVSTDRGALHKAISLEHAVHIIEETQLFQDFEPVQTLLLSSKKGNRFVYAGSNSGVVQAPLAFCGKHGTCEDCVLARDPYCAWSPPTATCVALHQTESPSRGLIQEMSGDASVCPDKSKGSYRQHFFKHGGTAELKCSQKSNLARVFWKFQNGVLKAESPKYGLMGRKNLLIFNLSEGDSGVYQCLSEERVKNKTVFQVVAKHVLEVKVVPKPVVAPTLSVVQTEGSRIATKVLVASTQGSSPPTPAVQATSSGAITLPPKPAPTGTSCEPKIVINTVPQLHSEKTMYLKSSDNRLLMSLFLFFFVLFLCLFFYNCYKGYLPRQCLKFRSALLIGKKKPKSDFCDREQSLKETLVEPGSFSQQNGEHPKPALDTGYETEQDTITSKVPTDREDSQRIDDLSARDKPFDVKCELKFADSDADGD

SEQ ID NO: 2 - protein, 862 aa, Mus sp.
MRMCAPVRGLFLALVVVLRTAVAFAPVPRLTWEHGEVGLVQFHKPGIFNYSALLMSEDKDTLYVGAREAVFAVNALNISEKQHEVYWKVSEDKKSKCAEKGKSKQTECLNYIRVLQPLSSTSLYVCGTNAFQPTCDHLNLTSFKFLGKSEDGKGRCPFDPAHSYTSVMVGGELYSGTSYNFLGSEPIISRNSSHSPLRTEYAIPWLNEPSFVFADVIQKSPDGPEGEDDKVYFFFTEVSVEYEFVFKLMIPRVARVCKGDQGGLRTLQKKWTSFLKARLICSKPDSGLVFNILQDVFVLRAPGLKEPVFYAVFTPQLNNVGLSAVCAYTLATVEAVFSRGKYMQSATVEQSHTKWVRYNGPVPTPRPGACIDSEARAANYTSSLNLPDKTLQFVKDHPLMDDSVTPIDNRPKLIKKDVNYTQIVVDRTQALDGTFYDVMFISTDRGALHKAVILTKEVHVIEETQLFRDSEPVLTLLLSSKKGRKFVYAGSNSGVVQAPLAFCEKHGSCEDCVLARDPYCAWSPAIKACVTLHQEEASSRGWIQDMSGDTSSCLDKSKESFNQHFFKHGGTAELKCFQKSNLARVVWKFQNGELKAASPKYGFVGRKHLLIFNLSDGDSGVYQCLSEERVRNKTVSQLLAYSHVLEVKMVPRTPPSPTSEDAQTEGSKITSKMPVASTQGSSPPTPALWATSPRAATLPPKSSSGTSCEPKMVINTVPQLHSEKTVYLKSSDNRLLMSLLLFIFVLFLCLFSYNCYKGYLPGQCLKFRSALLLGKKTPKSDFSDLEQSVKETLVEPGSFSQQNGDHPKPALDTGYETEQDTITSKVPTDREDSQRIDELSARDKPFDVKCELKFADSDADGD

SEQ ID NO: 3 - DNA, 30 bp, synthetic construct
  source 1..30: mol_type = other DNA
ggctacagcttcagcgactactacatgcac

SEQ ID NO: 4 - DNA, 51 bp, synthetic construct
  source 1..51: mol_type = other DNA
cagattaatcctaccactggcggcgctagctacaaccagaagttcaagggc

SEQ ID NO: 5 - DNA, 27 bp, synthetic construct
  source 1..27: mol_type = other DNA
tattactacggcagacacttcgatgtc

SEQ ID NO: 6 - protein, 10 aa, synthetic construct
GYSFSDYYMH

SEQ ID NO: 7 - protein, 17 aa, synthetic construct
QINPTTGGASYNQKFKG

SEQ ID NO: 8 - protein, 9 aa, synthetic construct
YYYGRHFDV

SEQ ID NO: 9 - protein, 118 aa, synthetic construct
QVQLVQSGAEVKKPGSSVKVSCKASGYSFSDYYMHWVRQAPGQGLEWMGQINPTTGGASYNQKFKGKATITVDKSTSTAYMELSSLRSEDTAVYYCARYYYGRHFDVWGQGTTVTVSS

SEQ ID NO: 10 - protein, 118 aa, synthetic construct
QVQLQQSGPELVKPGASVKISCKASGYSFSDYYMHWVKQSPENSLEWIGQINPTTGGASYNQKFKGKATLTVDKSSSTAYMQLKSLTSEESAVYYCTRYYYGRHFDVWGQGTTVTVSS

SEQ ID NO: 11 - DNA, 44 bp, synthetic construct
  source 1..44: mol_type = other DNA
aaggccagccaaagcgtggattatgatggcgatagctatatgaa

SEQ ID NO: 12 - DNA, 21 bp, synthetic construct
  source 1..21: mol_type = other DNA
gctgcatccaatctggaaagc

SEQ ID NO: 13 - DNA, 27 bp, synthetic construct
  source 1..27: mol_type = other DNA
cagcaaagcaatgaggatccctacacc

SEQ ID NO: 14 - protein, 15 aa, synthetic construct
KASQSVDYDGDSYMN

SEQ ID NO: 15 - protein, 7 aa, synthetic construct
AASNLES

SEQ ID NO: 16 - protein, 9 aa, synthetic construct
QQSNEDPYT

SEQ ID NO: 17 - protein, 111 aa, synthetic construct
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPYTFGQGTKLEIK

SEQ ID NO: 18 - protein, 111 aa, synthetic construct
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 19 - DNA, 354 bp, synthetic construct
  source 1..354: mol_type = other DNA
caggtgcagctggtgcagagcggcgctgaggtgaagaagcctggcagcagcgtgaaggtctcctgcaaggctagcggctacagcttcagcgactactacatgcactgggtgagacaggcccctggccaaggcctggagtggatgggccagattaatcctaccactggcggcgctagctacaaccagaagttcaagggcaaggccaccattaccgtggacaaaagcaccagcacagcctacatggagctgagcagcctgagaagcgaggacaccgccgtgtattactgtgccagatattactacggcagacacttcgatgtctggggccaaggcaccacggtcaccgtctcttca

SEQ ID NO: 20 - DNA, 354 bp, synthetic construct
  source 1..354: mol_type = other DNA
caggtccagctgcagcagtctggacctgagctggtgaagcctggggcttcagtgaagatatcctgcaaggcttctggttactcattcagtgactactacatgcactgggtgaagcaaagtcctgaaaatagtcttgagtggattggacagattaatcctaccactgggggtgctagctacaaccagaagttcaagggcaaggccacattaactgtagataaatcctccagcacagcctacatgcagctcaagagcctgacatctgaagagtctgcagtctattactgtacaagatattactacggtagacacttcgatgtctggggccaagggaccacggtcaccgtttcctca

SEQ ID NO: 21 - DNA, 333 bp, synthetic construct
  source 1..333: mol_type = other DNA
gacatcgtgatgacccagagcccagacagcctggctgtgagcctgggcgagagggccaccatcaactgcaaggccagccaaagcgtggattatgatggcgatagctatatgaactggtaccagcagaaaccaggccagcctcctaagctgctgatttacgctgcatccaatctggaaagcggcgtgcctgacagattcagcggcagcggcagcggcacagatttcactctgaccatcagcagcctgcaggctgaagatgtggcagtgtattactgtcagcaaagcaatgaggatccctacaccttcggccaagggaccaagctcgagatcaaa

SEQ ID NO: 22 - DNA, 333 bp, synthetic construct
  source 1..333: mol_type = other DNA
gacattgtgatgacccagtctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctcgagatcaaa

SEQ ID NO: 23 - protein, 118 aa, synthetic construct
QVQLQQSGAELAKPGASVKMSCKASGYTFTRYWMHWVKQRPGQGLEWIGYINPSTGYSDYNQKFKDKATLTADKSSSTAYMQLSSLTSEDSAVYYCARDPYGWTMDSWGQGTLVTVSS

SEQ ID NO: 24 - protein, 10 aa, synthetic construct
GYTFTRYWMH

SEQ ID NO: 25 - protein, 17 aa, synthetic construct
YINPSTGYSDYNQKFKD

SEQ ID NO: 26 - protein, 9 aa, synthetic construct
DPYGWTMDS

SEQ ID NO: 27 - protein, 107 aa, synthetic construct
DIQMTQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLEIK

SEQ ID NO: 28 - protein, 11 aa, synthetic construct
HASQNINVWLS

SEQ ID NO: 29 - protein, 7 aa, synthetic construct
KASNLHT

SEQ ID NO: 30 - protein, 9 aa, synthetic construct
QQGQSYPYT

SEQ ID NO: 31 - DNA, 354 bp, synthetic construct
  source 1..354: mol_type = other DNA
caggtccagctgcagcagtctggggctgaactggcaaaacctggggcctcagtgaagatgtcctgcaaggcttctggctacacctttactaggtactggatgcactgggtaaaacagaggcctggacagggtctggaatggattggatacattaatcctagcactggttattctgattacaatcagaagttcaaggacaaggccacattgactgcagacaaatcctccagcacagcctacatgcaactgagcagcctgacatctgaggactctgcagtctattactgtgcaagagacccctacggctggactatggactcctggggccaagggactctggtcaccgtctcctca

SEQ ID NO: 32 - DNA, 30 bp, synthetic construct
  source 1..30: mol_type = other DNA
ggctacacctttactaggtactggatgcac

SEQ ID NO: 33 - DNA, 51 bp, synthetic construct
  source 1..51: mol_type = other DNA
tacattaatcctagcactggttattctgattacaatcagaagttcaaggac

SEQ ID NO: 34 - DNA, 27 bp, synthetic construct
  source 1..27: mol_type = other DNA
gacccctacggctggactatggactcc

SEQ ID NO: 35 - DNA, 321 bp, synthetic construct
  source 1..321: mol_type = other DNA
gacatccagatgacccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtacacgttcggaggggggaccaagctcgagatcaaa

SEQ ID NO: 36 - DNA, 33 bp, synthetic construct
  source 1..33: mol_type = other DNA
catgccagtcagaacattaatgtttggttaagc

SEQ ID NO: 37 - DNA, 21 bp, synthetic construct
  source 1..21: mol_type = other DNA
aaggcttccaacttgcacaca

SEQ ID NO: 38 - DNA, 27 bp, synthetic construct
  source 1..27: mol_type = other DNA
caacagggtcaaagttatccgtacacg

SEQ ID NO: 39 - protein, 113 aa, synthetic construct
EVQLVESGGGLVQPGGSLRLSCAASGFIFSDYWMVWVRQAPGKGLEYVAHMNQDGGARYYAESVRGRFTISRDNAKNSLYLQMNSLRAEDTAVYYCARDPWGYDSWGQGTLVT

SEQ ID NO: 40 - protein, 5 aa, synthetic construct
DYWMV

SEQ ID NO: 41 - protein, 11 aa, synthetic construct
HMNQDGGARYY

SEQ ID NO: 42 - protein, 5 aa, synthetic construct
DPWGY

SEQ ID NO: 43 - protein, 104 aa, synthetic construct
SYELTQPPSVSVSPGQTASITCSGDKLGDKYAVWYQQKPGQSPVLVIYQDSKRPSGIPERFSGSNSGNTATLTISGTQAMDEADYYCQAWEQEAAWVFGGGTKL

SEQ ID NO: 44 - protein, 11 aa, synthetic construct
SGDKLGDKYAV

SEQ ID NO: 45 - protein, 7 aa, synthetic construct
QDSKRPS

SEQ ID NO: 46 - protein, 10 aa, synthetic construct
QAWEQEAAWV

SEQ ID NO: 47 - DNA, 20 bp, synthetic construct
  source 1..20: mol_type = other DNA
  modified_base 1: mod_base = OTHER; note = 2'-(2-methoxyethyl)ribose 5-methylcytosine phosphorothioate
  modified_base 2: mod_base = OTHER; note = 2'-(2-methoxyethyl)ribose nucleoside
  modified_base 3: mod_base = OTHER; note = 2'-(2-methoxyethyl)ribose 5-methylcytosine
  modified_base 4: mod_base = OTHER; note = 2'-(2-methoxyethyl)ribose nucleoside
  modified_base 5: mod_base = OTHER; note = 2'-(2-methoxyethyl)ribose nucleoside phosphorothioate
  modified_base 6: mod_base = OTHER; note = phosphorothioate linkage
  modified_base 7: mod_base = OTHER; note = phosphorothioate linkage
  modified_base 8: mod_base = OTHER; note = phosphorothioate linkage
  modified_base 9: mod_base = OTHER; note = 5-methylcytosine phosphorothioate
  modified_base 10: mod_base = OTHER; note = phosphorothioate linkage
  modified_base 11: mod_base = OTHER; note = phosphorothioate linkage
  modified_base 12: mod_base = OTHER; note = phosphorothioate linkage
  modified_base 13: mod_base = OTHER; note = phosphorothioate linkage
  modified_base 14: mod_base = OTHER; note = phosphorothioate linkage
  modified_base 15: mod_base = OTHER; note = 5-methylcytosine phosphorothioate
  modified_base 16: mod_base = OTHER; note = 2'-(2-methoxyethyl)ribose nucleoside
  modified_base 17: mod_base = OTHER; note = 2'-(2-methoxyethyl)ribose 5-methylcytosine
  modified_base 18: mod_base = OTHER; note = 2'-(2-methoxyethyl)ribose 5-methylcytosine
  modified_base 19: mod_base = OTHER; note = 2'-(2-methoxyethyl)ribose nucleoside phosphorothioate
  modified_base 20: mod_base = OTHER; note = 2'-(2-methoxyethyl)ribose 5-methylcytosine phosphorothioate
  misc_feature 1..5: note = RNA
  misc_feature 6..15: note = DNA
  misc_feature 16..20: note = RNA
ctcagtaacattgacaccac

SEQ ID NO: 48 - DNA, 20 bp, synthetic construct
  source 1..20: mol_type = other DNA
ctcagtaacattgacaccac

(19) United States
(12) Patent Application Publication
(10) Pub. No.: US 2023/0331863 A1
(43) Pub. Date: Oct. 19, 2023

(54) COMBINATION THERAPY WITH SEMAPHORIN-4D BLOCKADE AND HTT-LOWERING AGENT FOR TREATMENT OF HUNTINGTON'S DISEASE

(71) Applicants: VACCINEX, INC., Rochester, NY (US); UNIVERSITY OF CENTRAL FLOEIDA RESEARCH FOUNDATION, INC, Orlando, FL (US)

(72) Inventors: Elizabeth Evans, Bloomfield, NY (US); Terrence Fisher, Rochester, NY (US); Amber Southwell, Orlando, FL (US); Yuanyun Xie, Orlando, FL (US); Maurice Zauderer, Pittsford, NY (US)

(21) Appl. No.: 18/168,002

(22) Filed: Feb. 13, 2023

Related U.S. Application Data
(60) Provisional application No. 63/309,586, filed on Feb. 13, 2022.

(51) Int. Cl. C07K 16/28; C12N 15/113; A61P 25/28

(52) U.S. Cl. CPC C07K 16/2896; C12N 15/113; A61P 25/28; C07K 2317/565; C12N 2310/11

(57) ABSTRACT

Disclosed are combination therapies and methods for the treatment of Huntington's disease comprising the administration of a combination of a SEMA4D binding molecule and an HTT-lowering agent.

### CROSS-REFERENCE TO RELATED APPLICATIONS

The present application claims the benefit of priority under 35 U.S.C. § 119 to U.S. Provisional Application Ser. No. 63/309,586, titled “Combination Therapy With Semaphorin-4D Blockade and HTT-Lowering Agent For Treatment Of Huntington's Disease” and filed Feb. 13, 2022, the disclosure of which is incorporated herein by reference in its entirety.

### BACKGROUND OF THE DISCLOSURE

Huntington's disease (HD) is a progressive brain disorder caused by a single defective gene, the HTT gene on chromosome 4. The defect in the HTT gene, which encodes the huntingtin protein, is a CAG trinucleotide repeat expansion. The normal huntingtin gene includes 17 to 20 CAG repetitions. The fully penetrant defect that causes Huntington's disease includes 40 or more CAG repeats, a partially penetrant defect may occur with as few as 36 repeats.

The normal function of the huntingtin protein includes but may not be limited to acting as a transcriptional regulator during development and neurogenesis and serving as a scaffold protein and to signal stress in a variety of cellular processes. However, its defective form has been identified as the cause of HD. Defective huntingtin protein leads to brain changes that cause abnormal involuntary movements, a severe decline in thinking and reasoning skills, including memory, concentration, judgment, and ability to plan and organize and irritability, depression and other mood changes, especially apathy, depression, anxiety, and uncharacteristic anger and irritability. Another common symptom is obsessive-compulsive behavior, leading a person to repeat the same question or activity over and over. The hallmark symptom of early-stage Huntington's disease is uncontrolled movement of the arms, legs, head, face and upper body.

Although the exact function of the huntingtin protein is not fully understood, it is thought to play an important role in neurons, or nerve cells. The CAG trinucleotide repeat expansion in the gene results in a longer-than-usual huntingtin protein being produced. This abnormal protein is thought to form aggregates, which disrupt the normal function of neurons and their interactions with other brain cells that eventually cause their degeneration and death.

The neurons that are most sensitive to the mutated huntingtin protein, called medium spiny neurons, are found in areas of the brain called the striatum, a structure of the basal ganglia, and receive input from the cortex. These regions of the brain help to coordinate movement, thinking, and motivation, among other processes. When these neurons malfunction, those functions are disrupted, causing the signs and symptoms associated with HD.

There currently are no treatments that can alter the course of HD. Thus, there is a need for treatments that can lessen the symptoms, including movement and psychiatric symptoms of HD.

### BRIEF SUMMARY OF THE DISCLOSURE

This application addresses the need for safe and effective treatments for Huntington's disease that include a binding molecule that specifically binds to semaphorin-4D (SEMA4D) that acts in combination with an HTT-lowering therapy to enhance the benefit of each therapeutic agent to HD subjects. Disclosed are methods and compositions related to novel combination therapies comprising at least one inhibitor of semaphorin-4D (SEMA4D) and at least one HTT-lowering therapeutic agent.

In one aspect of the disclosure, there is provided a combination therapy for the treatment of Huntington's disease (HD) comprising at least one isolated antibody or antigen-binding fragment thereof that specifically binds to semaphorin-4D (SEMA4D) and a therapeutically effective amount of at least one HTT-lowering agent. In some embodiments, the antibody or antigen-binding fragment component of the combination therapy inhibits SEMA4D interactions with its receptor. In certain embodiments, the receptor is Plexin-B 1. In yet other embodiments, the antibody or antigen-binding fragment thereof inhibits SEMA4D-mediated Plexin-B 1 signal transduction. In certain embodiments the antibody or antigen-binding fragment thereof competitively inhibits a reference monoclonal antibody VX15/2503 or MAb67 from specifically binding to SEMA4D. In any of the embodiments of the combination therapy, the antibody or antigen-binding fragment thereof comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively. In other embodiments, the VH and VL comprise, respectively, SEQ ID NO: 9 and SEQ ID NO: 17 or SEQ ID NO: 10 and SEQ ID NO: 18. In further embodiments the at least one huntingtin (HTT)-lowering agent is an antisense oligonucleotide (ASO), such as an allele-selective ASO or a non-selective ASO. In some embodiments, the ASO and antibody or antigen-binding fragment thereof are administered separately or concurrently. In any of the embodiments of this aspect of the disclosure, administration of the combination of the isolated antibody or antigen-binding fragment thereof and the HTT-lowering agent results in enhanced therapeutic efficacy relative to administration of either the isolated antibody or antigen-binding fragment thereof or the HTT-lowering agent alone. In other embodiments, administration of the combination of the isolated antibody or antigen-binding fragment thereof and the HTT-lowering agent results in improvement of neuropsychiatric symptoms, cognitive symptoms, motor dysfunction, brain atrophy, metabolic activity, or any combination thereof. In certain embodiments, the improvement of neuropsychiatric symptoms is selected from the group consisting of reduced anxiety-like behavior, improved spatial memory, increased locomotion, and any combination thereof.

In another aspect of the disclosure, there is provided a method of treating a subject having Huntington's disease (HD) with a combination therapy comprising administering at least one isolated antibody or antigen-binding fragment thereof that specifically binds to semaphorin-4D (SEMA4D) and a therapeutically effective amount of at least one HTT-lowering agent. In some embodiments the antibody or antigen-binding fragment thereof inhibits SEMA4D interactions with its receptor. In certain embodiments, the SEMA4D receptor is Plexin-B1. In certain embodiments, the antibody or antigen-binding fragment thereof inhibits SEMA4D-mediated Plexin-B 1 signal transduction. In other embodiments of this aspect of the disclosure, the antibody or antigen-binding fragment thereof competitively inhibits a reference monoclonal antibody VX15/2503 or MAb67 from specifically binding to SEMA4D. In yet other embodiments, the antibody or antigen-binding fragment thereof comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively. In further embodiments, the VH and VL comprise, respectively, SEQ ID NO: 9 and SEQ ID NO: 17 or SEQ ID NO: 10 and SEQ ID NO: 18. In yet other embodiments of this aspect, the at least one huntingtin (HTT)-lowering agent is an antisense oligonucleotide (ASO), such as an allele-selective ASO or a non-selective ASO. In certain embodiments, the ASO is a non-selective ASO. In any of the embodiments of this aspect of the disclosure, the ASO and the antibody or antigen-binding fragment thereof are administered separately or concurrently. In any embodiment, administration of the combination of the isolated antibody or antigen-binding fragment thereof and the HTT-lowering agent results in enhanced therapeutic efficacy relative to administration of the isolated binding molecule or the immune modulating therapy alone. Administration of the combination of the isolated antibody or antigen-binding fragment thereof and the HTT-lowering agent results in improvement of neuropsychiatric symptoms, cognitive symptoms, motor dysfunction, brain atrophy, metabolic activity, or any combination thereof in any of the embodiments of this aspect of the disclosure. In certain embodiments, the improvement of neuropsychiatric symptoms is selected from the group consisting of reduced anxiety-like behavior, improved spatial memory, increased locomotion, and any combination thereof.

### BRIEF DESCRIPTION OF THE DRAWINGS

FIG. 1A is a graph of the average distance traveled in the Open Field Test at 3 months and 9 months by age-matched untreated wild type Hu18/18, untreated HD Hu97/18, single agent-treated (antisense oligonucleotide (HTT1 ASO) or anti-SEMA4D antibody (MAb67)), and combination therapy-treated (COMBO: ASO and MAb67) human huntingtin gene knock-in mice. FIG. 1B is a graph of the average velocity of the mice in the Open Field Test at the same time points.

FIG. 2A is a graph of the average number of entries into the center field made at 3 and 9 months by age-matched untreated wild type Hu18/18, untreated HD Hu97/18, single agent-treated (ASO or MAb67), and combination therapy-treated (COMBO—ASO and MAb67) mice in the Open Field Test at 3 and 9 months. FIG. 2B is a graph of the average amount of time the mice spent in the center field at the same time points. Reduced entries and time in center is thought to reflect an anxiety-like behavior.

FIG. 3A is a graph of the average number of entries to the open arms of an elevated plus maze away from the center made by untreated wild type Hu18/18, untreated HD Hu97/18, single agent-treated (ASO or MAb67), and combination therapy-treated (COMBO—ASO and MAb67) mice in the Elevated Maze Test with two open and two enclosed arms. FIG. 3B graphically demonstrates the average amount of time the mice spent in the open arms of the plus maze.

FIG. 4 is a graph showing the loss of striatal volume rescued by treatment of Hu97/18 mice treated with a combination of ASO and anti-SEMA4D antibody compared to single agent (ASO or anti-SEMA4D) treatment relative to control Hu97/18. A comparison of age-matched Hu18/18 and Hu97/18 control mice is also shown.

FIG. 5 is a graph showing the loss of cortical volume rescued by treatment of Hu97/18 mice treated with a combination of ASO and anti-SEMA4D antibody compared to single agent (ASO or anti-SEMA4D) treatment.

FIG. 6 is a graph showing the loss of corpus callosum volume rescued by treatment of Hu97/18 mice treated with a combination of ASO and anti-SEMA4D antibody compared to single agent (ASO or anti-SEMA4D) treatment.

FIG. 7A is a bar graph showing the effect of single agent anti-SEMA4D antibody (Mab67) and HH1 ASO treatment of Hu97/18 mice compared to combination treatment (Mab67+HTT1 ASO) on SEMA4D expression in NeuN+ neurons in the cortex. FIG. 7B shows the effect of single agent and combination treatment on SEMA4D expression in NeuN+ neurons in the Caudoputamen of Hu97/18 mice.

FIG. 8 is a graph showing the effect of single agent HT1 ASO and Mab67 treatment on level of mutant HTT protein (mtHTT) in plasma of Hu97/18 mice over time following treatment.

FIG. 9A-D are bar graphs showing the effect of single agent HH1 and MAb67 treatment and combination HH1 plus Mab67 treatment on reactive astrocytes in Hu18/18 and Hu97/18 mice via GFAP and Serpina3 staining. FIG. 9A: GFAP staining in the cortex. FIG. 9B: Serapin3 staining in the cortex. FIG. 9C: GFAP staining in the hypothalamus. FIG. 9D: Serapin3 staining in the hypothalamus. * p<0.05; ** p<0.01, *** p<0.001, **** p<0.0001.

### DETAILED DESCRIPTION OF THE DISCLOSURE

I. Definitions

In this specification and in the claims that follow, reference will be made to a number of terms which shall be defined to have the following meanings:

It is to be noted that the term “a” or “an” entity refers to one or more of that entity; for example, “an anti-SEMA4D antibody” is understood to represent one or more anti-SEMA4D antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

“Optional” or “optionally” means that the subsequently described event or circumstance may or may not occur, and that the description includes instances where said event or circumstance occurs and instances where it does not.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

The term “therapeutically effective amount” refers to an amount of an antibody, polypeptide, polynucleotide, small organic molecule, or other drug effective to “treat” a disease or disorder in a subject or mammal. In the case of a neurodegenerative disorder, the therapeutically effective amount of the drug can alleviate symptoms of the disorder; decrease, reduce, retard or stop the incidence of symptoms; decrease, reduce, retard the severity of symptoms; inhibit, e.g., suppress, retard, prevent, stop, or reverse the manifestation of symptoms; relieve to some extent one or more of the symptoms associated with the disorder; reduce morbidity and mortality; improve quality of life; or a combination of such effects.

The term “symptoms” as referred to herein refer to, e.g., 1) neuropsychiatric symptoms, 2) cognitive symptoms, 3) motor dysfunction, 4) brain atrophy and metabolic activity (e.g., cortex, corpus callosum, corpus striatum regions of the brain) and combinations thereof. Examples of neuropsychiatric symptoms include, for instance, anxiety-like behavior, sleep disturbances, and irritability. Examples of cognitive symptoms include, for instance, learning and memory deficits. Examples of motor dysfunction include, for instance, locomotion or coordination difficulties or repetitive movements, e.g., hand movements such as hand wringing.

Terms such as “treating” or “treatment” or “to treat” or “alleviating” or “to alleviate” or “improving” or “to improve” refer to both 1) therapeutic measures that cure, slow down, lessen symptoms of, reverse, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. Beneficial or desired clinical results include, but are not limited to, alleviation of symptoms, diminishment of extent of disease, stabilization (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already exhibiting symptoms of the condition or disorder as well as asymptomatic subjects.

By “subject” or “individual” or “animal” or “patient” or “mammal,” is meant any subject, particularly a mammalian subject, e.g., humans, for whom diagnosis, prognosis, or therapy is desired, such as a subject suspected of having, diagnosed with, or experiencing symptoms of Huntington's disease.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., anxiety or loss of motor control). It is understood that this is typically in relation to some standard or expected value, in other words it is relative, but that it is not always necessary for the standard or relative value to be referred to. For example, “reduces neuropsychiatric symptoms” or “reduces anxiety” means reducing the measurable level of a symptom such as anxiety relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, the term “healthcare provider” refers to individuals or institutions that directly interact and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

As used herein, the term “clinical laboratory” refers to a facility for the examination or processing of materials or images derived from a living subject, e.g., a human being. Non-limiting examples of processing include biological, biochemical, serological, chemical, immunohematological, hematological, biophysical, cytological, pathological, genetic, image based, or other examination of materials derived from the human body or of any or all of the human body for the purpose of providing information, e.g., for the diagnosis, prevention, or treatment of any disease or impairment of, or the assessment of the health of living subjects, e.g., human beings. These examinations can also include procedures to collect or otherwise obtain an image, a sample, prepare, determine, measure, or otherwise describe the presence or absence of various substances in the body of a living subject, e.g., a human being, or a sample obtained from the body of a living subject, e.g., a human being.

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

II. Combination Therapy

Huntingtin (HTT) lowering therapy has shown significant preclinical promise. However, HTT-lowering therapeutic intervention, particularly intervention that lowers both mutant and wild-type HTT, may not be sufficient to prevent further damage and is unlikely to repair the substantial damage to neurons and brain cells that has already occurred at time of treatment. These potential limitations suggest that therapeutic strategies that combine HTT-lowering intervention with independent strategies to promote health and recovery of the damaged brain could provide more comprehensive benefit.

Anti-semaphorin4D (SEMA4D) immunotherapy has great potential for combination therapy to treat HD, as its target, SEMA4D, is elevated in brains of HD patients, and preliminary studies indicate that it may prevent loss of protective glial functions and restore vascular changes associated with neuronal dysfunction and degeneration in HD. Our studies have demonstrated significant benefit from anti-SEMA4D immunotherapy in terms of the HD cognitive assessment battery composite score as well as reduced caudate atrophy and increased brain metabolic activity in individuals with early manifest disease. (U.S. Pat. No. 9,598,495). Similarly, we have previously demonstrated preclinical benefit of an anti-SEMA4D monoclonal antibody (MAb67) in YAC128 HD model mice preventing grey and white matter loss and reducing some cognitive deficits. (Southwell et al., Anti-semaphorin-4D immunotherapy ameliorates neuropathology and some cognitive impairment in the YAC128 mouse model of Huntington disease, (2015) Neurbiol. Dis., 76:46-56). These findings support the benefit of the disclosed combination anti-SEMA4D immunotherapy and HTT-lowering therapy for the treatment of HD.

III. Combination Therapy Components: HTT-Lowering Agents and Anti-SEMA4D Immuno-Therapies

Huntington's disease (HD) is a rare genetic neurodegenerative disease characterized by cognitive, behavioral, and motor symptoms. (Bates G P, Dorsey R, Gusella J F, et al. Huntington disease. Nat Rev Dis Primers. 2015; 1:15005; Roos R A C. Huntington disease: a clinical review. Orphanet J Rare Dis. 2010; 5(5):40; Ross C A, Aylward E H, Wild E J, et al., Huntington disease: natural history, biomarkers and prospects for therapeutics. Nat Rev Neurol. 2014; 10(4):204-216). Disease onset typically occurs in the prime of life, between ages 30 and 50 years, and is associated with increasing disability, worsening of function, and loss of independence, leading to death within approximately 15 years, on average, after the onset of motor signs and symptoms. (Roos R A C. Huntington disease: a clinical review. Orphanet J Rare Dis. 2010; 5(5):40, 1750-1172-5-402; Keum J W, Shin A, Gillis T, et al.)

The HTT gene encodes the protein, huntingtin (HTT). Although the exact function of this protein is unknown, it appears to play an important role in neurons in the brain and is essential for normal development before birth. Huntingtin is found in many of the body's tissues, with the highest levels of activity in the brain. One region of the HTT gene contains a particular DNA segment known as a CAG trinucleotide repeat. This segment is made up of a series of cytosine, adenine, and guanine (CAG) that appear multiple times in a row. Normally, the CAG segment is repeated 10 to 35 times within the gene. A cytosine-adenine-guanine (CAG) repeat expansion mutation in only 1 of the 2 alleles of the HTT gene is sufficient to be associated with the onset of HD with an autosomal-dominant pattern of inheritance. (Squitieri F, Gellera C, Cannella M, et al. Homozygosity for CAG mutation in Huntington disease is associated with a more severe clinical course. Brain. 2003; 126 (pt 4):946-955). The expansion variation on the affected allele is encoded for an abnormally long polyglutamine tract within the huntingtin protein (HTT), resulting in the formation of variant HTT protein. (The Huntington's Disease Collaborative Research Group. A novel gene containing a trinucleotide repeat that is expanded and unstable on Huntington's disease chromosomes. Cell, 1993; 72(6):971-983); Lee J-M, Ramos E M, Lee J-H, et al; PREDICT-HD Study of the Huntington Study Group (HSG); REGISTRY Study of the European Huntington's Disease Network; HD-MAPS Study Group; COHORT Study of the HSG CAG repeat expansion in Huntington disease determines age at onset in a fully dominant fashion. Neurology, 2012; 78(10): 690-695). The expression of mutant HTT throughout the brain is associated with progressive age dependent neurodegeneration, primarily owing to toxic gain-of-function mechanisms. (Nopoulos P C. Huntington disease: a single-gene degenerative disorder of the striatum. Dialogues Clin Neurosci. 2016; 18(1):91-98; Cattaneo E, Zuccato C, Tartan M. Normal huntingtin function: an alternative approach to Huntington's disease. Nat Rev Neurosci. 2005; 6(12):919-930).

HTT-Lowering Agents

The term “HTT-lowering agent” as used herein means a therapeutic agent or therapy aimed at silencing or repairing the mutant HTT gene to thereby lower the amount of mutant HTT protein in a subject with HD. Such agents and strategies include, but are not limited to, RNA interference (RNAi); antisense oligonucleotides (ASOs); ribozymes; DNAzymes, which are a class of single-stranded catalytic nucleic acids that bind to complementary mRNAs transcripts; DNA enzymes; and genome-editing approaches, e.g., gene therapy, CRISPR-Cas9. Various HTT-lowering agents and therapies that are useful in the methods and compositions disclosed herein are discussed below.

Approaches designed to interact with and decrease HTT mRNA include antisense oligonucleotides (ASOs), which are single stranded DNA molecules targeted to the HTT gene and RNA interference (RNAi) compounds that accelerate degradation of the HTT transcript; and orally bioavailable small molecules to reduce HTT through altering mRNA splicing. Agents that interact directly with HTT DNA include zinc finger transcriptional repressors (ZFTRs) and CRISPR/Cas9 ‘genome editing’ constructs. (Wild, E. J. and S. J. Tabrizi (2017), “Therapies targeting DNA and RNA in Huntington's disease.” Lancet Neurol 16(10): 837-847).

ASOs have a more upstream site of action than RNAi effectors. They are short, synthetic, single-stranded oligonucleotide analogs having ˜16-22 bases that bind to complementary pre-mRNA targets, e.g., HTT mRNA in the nucleus through Watson-Crick base-pairing and can lead to the modulation of gene expression through a number of potential pathways. One such pathway is through RNase H1 recruitment. Upon ASO binding, an RNA-DNA hybrid is formed that becomes a substrate for RNase H1, which degrades the target mRNA through hydrolysis. Cleavage products are then cleared through normal cellular mechanisms in the nucleus and cytoplasm. Other, non-limiting examples of potential pathways for ASO modulation of HTTgene expression include:

ASO binding to the AUG translation start site of HTT mRNA to cause steric hindrance of the ribosomal machinery and translational arrest; ASO binding to HTT intron-exon junctions to modulate splicing and could potentially inhibit the formation of alternatively spliced HTT exon 1 mRNA; ASOs that incorporate ribozymes or DNAzymes could also directly cleave the target HTT mRNA after hybridization.

ASOs that are useful HTT-lowering agents in the methods and combination therapies described herein include ASOs that are either allele-selective in order to specifically target the HTT mutation (muHTT) (SNP-targeted) or non-selective and thereby bind to both mutant and wild-type HTT. Both allele-selective and non-selective HTT ASOs are useful in the disclosed combination therapies. The ASO known as tominersen or HTTR_{X} (previously referred to as RG6042) and ISIS 443139 (Ionis Pharmaceuticals) is an example of a non-selective ASO that is designed to reduce the production of all forms of the huntingtin protein, including both its wild-type and mutant variant. (Tabrizi, B. et al., Effects of IONIS-HTTRx in patients with early Huntington's disease, results of the first HTT-lowering drug trial (CT.002) Neurology (2018)) Tominersen is a chemically modified synthetic oligonucleotide that is perfectly complementary to a 20-nucleotide stretch of HTT mRNA. HTTRx binds to HTT mRNA by means of Watson-Crick base pairing, with hybridization resulting in endogenous RNase H1-mediated degradation of the HTT mRNA, thus inhibiting translation of the huntingtin protein. The sequence of HTTRx is (5′ to 3′) ctₒcₒaₒgTAACATTGACaₒcₒcₒac (SEQ ID NO: 47), in which capital letters represent 2′-deoxyribose nucleosides, and small letters 2′-(2-methoxyethyl)ribose nucleosides. Nucleoside linkages that are represented with a subscripted “o” are phosphodiester, and all others are phosphorothioate. Letters “a” and “A” represent adenine, “c” and “C” 5-methylcytosine, “g” and “G” guanine, and “t” and “T” thymine nucleobases. (Tabrizi S J, Leavitt B R, Landwehrmeyer G B, Wild E J, Saft C, Barker R A, Blair N F, Craufurd D, Priller J, Rickards H, Rosser A, Kordasiewicz H B, Czech C, Swayze E E, Norris D A, Baumann T, Gerlach I, Schobel S A, Paz E, Smith A V, Bennett C F, Lane R M; Phase 1-2a IONIS-HTTRx Study Site Teams. Targeting Huntingtin Expression in Patients with Huntington's Disease. N Engl J Med. (2019) 380(24):2307-2316).

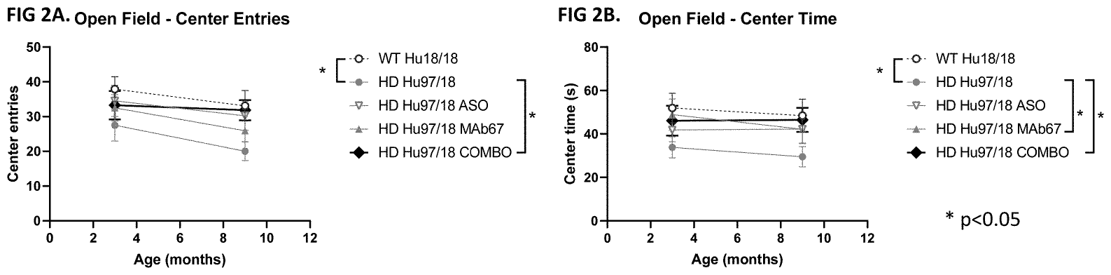

The ASO designated HH1 in the disclosed combination therapy is an ASO having the identical nucleotide sequence, CTCAGTAACATTGACACCAC, as HTTRx (SEQ ID NO: 48) described above (Southwell, A. L. et al: In vivo evaluation of candidate allele-specific mutant huntingtin gene silencing antisense oligonucleotides. Mol Ther (2014) 22:2093-2106, which is incorporated by reference herein). The sequence and properties of other non-selective and selective ASOs that could also be used in the combination therapy of the disclosure are described in Southwell, (ibid, FIG. 2, incorporated herein by reference). In a screen of a large panel of HTT-lowering ASOs, Southwell demonstrated that ASOs with at least 34 unique sequences, including HH1 (SEQ ID NO: 48) employed in the disclosed combination therapy, are effective at target engagement and lowering HTT. Between 76 and 85% knock down of muHTT protein was observed after treatment with pan species-HTT or Human-HTT ASOs. Treatment with allele-selective ASOs resulted in muHTT knock down between 28 and 94%. Several HTT lowering preclinical trials in rodents, treating with both selective and non-selective ASOs have shown significant benefit to a broad range of HD-like phenotypes. In preclinical studies, 50% suppression of mtHTT appears to be sufficient to provide benefit (Kordasiewicz Holly B, Stanek Lisa M, Wancewicz Edward V, Mazur C, McAlonis Melissa M, Pytel Kimberly A, et al. Sustained Therapeutic Reversal of Huntington's Disease by Transient Repression of Huntingtin Synthesis. Neuron. 2012; 74(6):1031-44.) and total HTT can be lowered by 50% without overt phenotypic change (Duyao M, Auerbach A, Ryan A, Persichetti F, Barnes G, McNeil S, et al. Inactivation of the mouse Huntington's disease gene homolog Hdh. Science. 1995; 269(5222):407-10). One study demonstrated that the benefit of HTT lowering outlasted the duration of HTT biochemical reduction (Kordasiewicz, H B et al. Sustained therapeutic reversal of Huntington's disease by transient repression of Huntingtin synthesis. (2012) Neuron 74: 1031-1044). Reduction of HTT in preclinical and clinical studies is considered a key biomarker of drug activity.

Allele-selective ASOs that are useful in the combination therapy of the disclosure and that specifically target single nucleotide polymorphisms (SNPs) of mutant HTT, but because not all patients have the sameSNPs, specific allele selective ASOs cannot be used to treat all HD patients. Such ASOs can be used, for example, in patients whose HTT mutation is complementary to the allele-selective ASO sequence. Examples of HD allele-selective ASOs include ASO WVE-120101, WVE-120102, and WVE-003 (Svrzikapa et al., Investigational Assay for Haplotype Phasing of the Huntington Gene, Mol. Ther., (2020) 19:162-172; incorporated herein by reference). These ASOs are expected to have a population coverage of WVE-120101: 20-56%, WVE-120102: 24-51%, and combinatorial use is expected to cover 36-70% of the HD patient population (Kay C, Collins J A, Caron N S, Agostinho Ld A, Findlay-Black H, Casal L, et al. A Comprehensive Haplotype Targeting Strategy for Allele-Specific HTT Suppression in Huntington Disease. The American Journal of Human Genetics. (2019) 105(6):1112-1125).

In addition to having different sequences that selectively target HD-associated SNPs or non-selectively target both mutant and wild-type HTT genes, the chemistry of the HTT ASO can be manipulated to increase efficacy and suitability as a therapeutic agent. For example, substitution of sulfur for non-bridging oxygen atoms to generate a phosphorothioate (PS) backbone provides nuclease resistance and improved protein binding as well as an increased half-life. In addition, multiple alterations at the 2′ position of the ribose sugar moiety have led to improved safety and efficacy of ASOs with increased binding affinity to the target mRNA, further resistance to nucleases, and decreased immunogenicity (Rinaldi and Wood, Antisense oligonucleotides: the next frontier for treatment of neurological disorders, Nat. Rev. Neurol., (2018) 14: 9-21 2018). For example, the HTTRx ASO incorporates both phosphorothioate and 2′-O-methoxyethyl (sugar) modifications. (Southwell, A. L. et al: In vivo evaluation of candidate allele-specific mutant huntingtin gene silencing antisense oligonucleotides. Mol Ther (2014) 22:2093-2106) In most ASOs, enhanced stability is accomplished by replacing the oxygen in the phosphodiester linkage with a sulfur to create a phosphorothioate (PS) linkage that is slightly more resistant to endonuclease activity [Shen et al. Chemistry, mechanism and clinical status of anti-sense oligonucleotides and duplex RNAs ((2018) Nucleic Acids Res., 46(4): 1584-600).

Beyond stability, PS linkages enhance ASO distribution by forming disulfide bonds with albumin, the most abundant protein in blood plasma and the cerebrospinal fluid (CSF), which transports the ASO throughout the CNS (Crooke et al., Phosphorothioate modified oligonucleotide-protein interactions, Nuc. Acids Res. (2020) 48(10): 5235-33; LaVine et al. Albumin and multiple sclerosis (2016) BMC Neurol. 16:47). Aside from benefits to stability and distribution, PS linkages can cause immune activation. However, incorporating phosphodiester (PO) linkages into the sequence creating a mixed (PS/PO) backbone can minimize this response. (Zhou, et al., Mixed-Backbone oligonucleotides as second-generation antisense agents with reduced phosphorothioate-related side effects, Bioorganic & Medicinal Chem Letts. (1998) 8(22): 3269-74); Pisetsky et al., Influence of backbone chemistry on immune activation by synthetic oligonucleotides (1999) Biochemical Pharmacology, 58(12): 1981-8. Both the HTTRx and Wave ASOs have (PS/PO) mixed backbones.

The methods and combination therapies disclosed herein also contemplate the use of DNA-targeting therapeutics, including those mediated by alternative splicing and possible gene-editing to lower HTT in a subject with HD. Gene therapy approaches to lowering HTT include delivery of siRNA or miRNA, via a viral vector, such as an adeno-associated virus (AAV) or lentivirus vector (Keiser, M. S., et al. (2016). “Gene suppression strategies for dominantly inherited neurodegenerative diseases: lessons from Huntington's disease and spinocerebellar ataxia.” Hum Mol Genet 25(R1): R53-64.). Generally, such vectors are administered by surgical injection into the brain parenchyma. This mode of administration should permit lifelong treatment from a single dose. Another gene therapy approach to lowering HTT includes the use of the CRISPR-Cas9 system to inactivate mutant HTT genes. (Dabrowska, et al. (2018), Precise Excision of the CAG Tract from the Huntingtin Gene by Cas9 Nickases, Neurosci, 12:75). Yet another DNA targeting approach to lowering HTT is the use of zinc finger proteins (ZFP) that are tooled for therapeutic use. For example, a zinc finger array specific to the longer CAG repeat of the mutant HTT gene, can be used to reduce chromosomal expression of the mutant gene. (Canut et al., Synthetic zinc finger repressors reduce mutant huntingtin expression in the brain of R6/2 mice, Proc Natl Acad Sci USA. (2012) 109:45). Brain-penetrant small molecules that alter splicing of HTT can also be used to lower HTT in a HD subject.

RNA interference (RNAi) is another useful approach for lowering HTT in the methods and compositions described herein. (Wild, E. J. and S. J. Tabrizi (2017). “Therapies targeting DNA and RNA in Huntington's disease.” Lancet Neurol 16(10): 837-847; Godinho, B. M., et al. (2015). “Delivering a disease-modifying treatment for Huntington's disease.” Drug Discov Today 20(1): 50-64). RNAi interferes with an endogenous cellular pathway that enables post-transcriptional regulation of expression of a target gene, such as the HTT gene. RNAi regulates gene expression by a highly precise mechanism of sequence-directed gene silencing at the stage of translation by degrading specific messenger RNAs or by blocking its translation into protein. (Godinho, B. M., et al. (2015))

Post-transcriptional gene silencing using ribozymes and DNA enzymes (DNAzymes) that are aimed at the elimination or repair of target mRNA transcripts may also be used to lower HTT in an HD subject. (Godinho, B. M., et al. (2015). “Delivering a disease-modifying treatment for Huntington's disease.” Drug Discov Today 20(1): 50-64). Transcription activator-like effector nucleases (TALEN) (Godinho, B. M., et al. (2015)) and transcription activator-like effectors (TALE) (Fink, K. D., et al. (2016), “Allele-Specific Reduction of the Mutant Huntingtin Allele Using Transcription Activator-Like Effectors in Human Huntington's Disease Fibroblasts.” Cell Transplant 25(4): 677-686) can also be used to lower HTT protein.

Another strategy for lowering HTT in an HD subject useful in the methods and compositions described herein is the use of brain-penetrant small molecules that alter splicing of HTT. (Bhattacharyya et al., Small molecule splicing modifiers with systemic HTT-lowering activity, Nature Comm., (2021), 12:7299) and Branaplam (Yu A M, Choi Y H, Tu M J. RNA Drugs and RNA Targets for Small Molecules: Principles, Progress, and Challenges. Pharmacol Rev. (2020) 72(4):862-898) (Novartis).

These and other compounds for lowering HTT are useful in the disclosed combination therapies and methods.

### Semaphorin-4D Binding Molecules

A “binding molecule” or “antigen binding molecule” of the present disclosure refers in its broadest sense to a molecule that specifically binds an antigenic determinant. In one embodiment, the binding molecule specifically binds to SEMA4D, e.g., to a transmembrane SEMA4D polypeptide of about 150 kDa or a soluble SEMA4D polypeptide of about 120 kDa (commonly referred to as sSEMA4D). In another embodiment, a binding molecule of the disclosure is an antibody or an antigen binding fragment thereof. In another embodiment, a binding molecule of the disclosure comprises at least one heavy or light chain CDR of an antibody molecule. In another embodiment, a binding molecule of the disclosure comprises at least two CDRs from one or more antibody molecules. In another embodiment, a binding molecule of the disclosure comprises at least three CDRs from one or more antibody molecules. In another embodiment, a binding molecule of the disclosure comprises at least four CDRs from one or more antibody molecules. In another embodiment, a binding molecule of the disclosure comprises at least five CDRs from one or more antibody molecules. In another embodiment, a binding molecule of the disclosure comprises at least six CDRs from one or more antibody molecules.

Unless specifically referring to full-sized antibodies such as naturally occurring antibodies, the term“anti-SEMA4D antibody” encompasses full-sized antibodies as well as antigen-binding fragments, variants, analogues, or derivatives of such antibodies, e.g., naturally occurring antibody or immunoglobulin molecules or engineered antibody molecules or fragments that bind antigen in a manner similar to antibody molecules.

As used herein, “human” or “fully human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example, in U.S. Pat. No. 5,939,598 by Kucherlapati et al. “Human” or “fully human” antibodies also include antibodies comprising at least the variable domain of a heavy chain, or at least the variable domains of a heavy chain and a light chain, where the variable domain(s) have the amino acid sequence of human immunoglobulin variable domain(s).

“Human” or “fully human” antibodies also include “human” or “fully human” antibodies, as described above, that comprise, consist essentially of, or consist of, variants (including derivatives) of antibody molecules (e.g., the VH regions and/or VL regions) described herein, which antibodies or fragments thereof immunospecifically bind to a SEMA4D polypeptide or fragment or variant thereof. Standard techniques known to those of skill in the art can be used to introduce mutations in the nucleotide sequence encoding a human anti-SEMA4D antibody, including, but not limited to, site-directed mutagenesis and PCR-mediated mutagenesis which result in amino acid substitutions. In some embodiments, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3.

In certain embodiments, the amino acid substitutions are conservative amino acid substitutions, discussed further below. Alternatively, mutations can be introduced randomly along all or part of the coding sequence, such as by saturation mutagenesis, and the resultant mutants can be screened for biological activity to identify mutants that retain activity (e.g., the ability to bind a SEMA4D polypeptide, e.g., human, murine, or both human and murine SEMA4D). Such variants (or derivatives thereof) of “human” or “fully human” antibodies can also be referred to as human or fully human antibodies that are “optimized” or “optimized for

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al. (1988) Antibodies: A Laboratory Manual (2nd ed.; Cold Spring Harbor Laboratory Press).

As used herein, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1- γ4 γ4.γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgA1, IgA2, etc. are well characterized and are known to confer functional specialization. Modified versions of each of these classes and isotypes are readily discernible to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (K, λ) Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VL or VK) and heavy (VH) chain portions determine antigen recognition and specificity. Conversely, the constant domains of the light chain (CL) and the heavy chain (typically CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 and CL domains typically comprise the carboxy-terminus of the heavy and light chain, respectively.

As indicated above, the variable region allows the antibody to selectively recognize and specifically bind epitopes on antigens. That is, the VL domain and VH domain, or subset of the complementarity determining regions (CDRs) within these variable domains, of an antibody combine to form the variable region that defines a three-dimensional antigen binding site. This quaternary antibody structure forms the antigen binding site present at the end of each arm of the Y. More specifically, the antigen binding site is defined by three CDRs on each of the VH and VL chains. In some instances, e.g., certain immunoglobulin molecules derived from camelid species or engineered based on camelid immunoglobulins, a complete immunoglobulin molecule can consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three-dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see below).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al. (1983) U.S. Dept. of Health and Human Services, “Sequences of Proteins of Immunological Interest” and by Chothia and Lesk, J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions ¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹ Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest.” Unless otherwise specified, references to the numbering of specific amino acid residue positions in an anti-SEMA4D antibody or antigen-binding fragment, variant, or derivative thereof of the present disclosure are according to the Kabat numbering system.

Antibodies or antigen-binding fragments, variants, or derivatives thereof of the disclosure include, but are not limited to, polyclonal, monoclonal, multispecific and bispecific in which at least one arm is specific for SEMA4D, human, humanized, primatized, or chimeric antibodies, single-chain antibodies, epitope-binding fragments, e.g., Fab, Fab′ and F(ab′) 2, Fd, Fvs, single-chain Fvs (scFv), disulfide-linked Fvs (sdFv), fragments comprising either a VL or VH domain, fragments produced by a Fab expression library, and anti-idiotypic (anti-Id) antibodies (including, e.g., anti-Id antibodies to anti-SEMA4D antibodies disclosed herein). ScFv molecules are known in the art and are described, e.g., in U.S. Pat. No. 5,892,019. Immunoglobulin or antibody molecules of the disclosure can be of any type (e.g., IgG, IgE, IgM, IgD, IgA, and IgY), class (e.g., IgG1, IgG2, IgG3, IgG4, IgA1, and IgA2, etc.), or subclass of immunoglobulin molecule.

As used herein, the term “heavy chain portion” includes amino acid sequences derived from an immunoglobulin heavy chain. In certain embodiments, a polypeptide comprising a heavy chain portion comprises at least one of: a VH domain, a CH1 domain, a hinge (e.g., upper, middle, and/or lower hinge region) domain, a CH2 domain, a CH3 domain, or a variant or fragment thereof. For example, a binding polypeptide for use in the disclosure can comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, a binding polypeptide for use in the disclosure can lack at least a portion of a CH2 domain (e.g., all or part of a CH2 domain). As set forth above, it will be understood by one of ordinary skill in the art that these domains (e.g., the heavy chain portions) can be modified such that they vary in amino acid sequence from the naturally occurring immunoglobulin molecule.

In certain anti-SEMA4D antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the disclosure are not identical. For example, each monomer can comprise a different target binding site, forming, for example, a bispecific antibody.

The heavy chain portions of a binding molecule for use in the methods disclosed herein can be derived from different immunoglobulin molecules. For example, a heavy chain portion of a polypeptide can comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain, e.g., a kappa or lambda light chain. In some aspects, the light chain portion comprises at least one of a VL or CL domain.

Anti-SEMA4D antibodies, or antigen-binding fragments, variants, or derivatives thereof disclosed herein can be described or specified in terms of the epitope(s) or portion(s) of an antigen, e.g., a target polypeptide disclosed herein (e.g., SEMA4D) that they recognize or specifically bind. The portion of a target polypeptide that specifically interacts with the antigen binding domain of an antibody is an “epitope,” or an “antigenic determinant.” A target polypeptide can comprise a single epitope, but typically comprises at least two epitopes, and can include any number of epitopes, depending on the size, conformation, and type of antigen. Furthermore, it should be noted that an “epitope” on a target polypeptide can be or can include non-polypeptide elements, e.g., an epitope can include a carbohydrate side chain.

The minimum size of a peptide or polypeptide epitope for an antibody is thought to be about four to five amino acids. Peptide or polypeptide epitopes can contain, e.g., at least seven, at least nine or between at least about 15 to about 30 amino acids. Since a CDR can recognize an antigenic peptide or polypeptide in its tertiary form, the amino acids comprising an epitope need not be contiguous, and in some cases, can be on separate peptide chains. A peptide or polypeptide epitope recognized by anti-SEMA4D antibodies of the present disclosure can contain a sequence of at least 4, at least 5, at least 6, at least 7, at least 8, at least 9, at least 10, at least 15, at least 20, at least 25, or between about 15 to about 30 contiguous or non-contiguous amino acids of SEMA4D.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” can be deemed to have a higher specificity for a given epitope than antibody “B,” or antibody “A” can be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody that “preferentially binds” to a given epitope would more likely bind to that epitope than to a related epitope, even though such an antibody can cross-react with the related epitope.

By way of non-limiting example, an antibody can be considered to bind a first epitope preferentially if it binds the first epitope with a dissociation constant (KD) that is less than the antibody's KD for the second epitope. In another non-limiting example, an antibody can be considered to bind a first antigen preferentially if it binds the first epitope with an affinity that is at least one order of magnitude less than the antibody's KD for the second epitope. In another non-limiting example, an antibody can be considered to bind a first epitope preferentially if it binds the first epitope with an affinity that is at least two orders of magnitude less than the antibody's KD for the second epitope.

In another non-limiting example, an antibody can be considered to bind a first epitope preferentially if it binds the first epitope with an off rate (k(off)) that is less than the antibody's k(off) for the second epitope. In another non-limiting example, an antibody can be considered to bind a first epitope preferentially if it binds the first epitope with an affinity that is at least one order of magnitude less than the antibody's k(off) for the second epitope. In another non-limiting example, an antibody can be considered to bind a first epitope preferentially if it binds the first epitope with an affinity that is at least two orders of magnitude less than the antibody's k(off) for the second epitope. An antibody or antigen-binding fragment, variant, or derivative disclosed herein can be said to bind a target polypeptide disclosed herein (e.g., SEMA4D, e.g., human, murine, or both human and murine SEMA4D) or a fragment or variant thereof with an off rate (k(off)) of less than or equal to 5×10⁻² sec⁻¹, 10⁻² sec⁻¹, 5×10−3 sec⁻¹ or 10⁻³ sec⁻¹. In certain aspects, an antibody of the disclosure can be said to bind a target polypeptide disclosed herein (e.g., SEMA4D, e.g., human, murine, or both human and murine SEMA4D) or a fragment or variant thereof with an off rate (k(off)) less than or equal to 5×10⁻⁴ sec−1, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein can be said to bind a target polypeptide disclosed herein (e.g., SEMA4D, e.g., human, murine, or both human and murine SEMA4D) or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. In some embodiments, an antibody of the disclosure cab be said to bind a target polypeptide disclosed herein (e.g., SEMA4D, e.g., human, murine, or both human and murine SEMA4D) or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al. (1988) Antibodies: A Laboratory Manual (Cold Spring Harbor Laboratory Press, 2nd ed.) pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valences of the immunoglobulins and the antigen. For example, the interaction between a bivalent monoclonal antibody and an antigen with a highly repeating epitope structure, such as a polymer, would be one of high avidity.

Anti-SEMA4D antibodies or antigen-binding fragments, variants, or derivatives thereof of the disclosure can also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross-reactive epitope generally contains many of the same complementary structural features as the inducing epitope, and in some cases, can actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody can be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody can be deemed “highly specific” for a certain epitope, if it does not bind any other analogue, ortholog, or homolog of that epitope.

Anti-SEMA4D binding molecules, e.g., antibodies or antigen-binding fragments, variants or derivatives thereof, of the disclosure can also be described or specified in terms of their binding affinity to a polypeptide of the disclosure, e.g., SEMA4D, e.g., human, murine, or both human and murine SEMA4D. In certain aspects, the binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10 M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In certain embodiments, the anti-SEMA4D binding molecule, e.g., an antibody or antigen binding fragment thereof, of the disclosure binds human SEMA4D with a Kd of about 5×10⁻⁹ to about 6×10⁻⁹. In another embodiment, the anti-SEMA4D binding molecule, e.g., an antibody or antigen binding fragment thereof, of the disclosure binds murine SEMA4D with a Kd of about 1×10⁻⁹ to about 2×10⁻⁹.

As used herein, the term “chimeric antibody” means any antibody wherein the immunoreactive region or site is obtained or derived from a first species and the constant region (which can be intact, partial or modified in accordance with the instant disclosure) is obtained from a second species. In certain embodiments the target binding region or site will be from a non-human source (e.g., mouse or primate) and the constant region is human.

As used herein, the term “engineered antibody” refers to an antibody in which the variable domain in either the heavy or light chain or both is altered by at least partial replacement of one or more CDRs from an antibody of known specificity and, if necessary, by partial framework region replacement and sequence changing. Although the CDRs can be derived from an antibody of the same class or even subclass as the antibody from which the framework regions are derived, it is envisaged that the CDRs will be derived from an antibody of different class, or from an antibody from a different species. An engineered antibody in which one or more “donor” CDRs from a non-human antibody of known specificity is grafted into a human heavy or light chain framework region is referred to herein as a “humanized antibody.” It is not always necessary to replace all of the CDRs with the complete CDRs from the donor variable domain to transfer the antigen binding capacity of one variable domain to another. Rather, one can transfer just those residues needed to maintain the activity of the target binding site need be transferred.

It is further recognized that the framework regions within the variable domain in a heavy or light chain, or both, of a humanized antibody can comprise solely residues of human origin, in which case these framework regions of the humanized antibody are referred to as “fully human framework regions” (for example, MAbs VX15/2503 or 67, disclosed in U.S. Patent Appl. Publication No. US 2010/0285036 A1 as MAb 2503, incorporated herein by reference in its entirety; and MAb 2517, disclosed in U.S. Patent Appl. Publication No. 2021/0032329, incorporated herein in its entirety by reference). Alternatively, one or more residues of the framework region(s) of the donor variable domain can be engineered within the corresponding position of the human framework region(s) of a variable domain in a heavy or light chain, or both, of a humanized antibody if necessary to maintain proper binding or to enhance binding to the SEMA4D antigen. A human framework region that has been engineered in this manner would thus comprise a mixture of human and donor framework residues and is referred to herein as a “partially human framework region.”

For example, humanization of an anti-SEMA4D antibody can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent or mutant rodent CDRs or CDR sequences for the corresponding sequences of a human anti-SEMA4D antibody. See also U.S. Pat. Nos. 5,225,539; 5,585,089; 5,693,761; 5,693,762; 5,859,205; herein incorporated by reference. The resulting humanized anti-SEMA4D antibody would comprise at least one rodent or mutant rodent CDR within the fully human framework regions of the variable domain of the heavy and/or light chain of the humanized antibody. In some instances, residues within the framework regions of one or more variable domains of the humanized anti-SEMA4D antibody are replaced by corresponding non-human (for example, rodent) residues (see, for example, U.S. Pat. Nos. 5,585,089; 5,693,761; 5,693,762; and 6,180,370), in which case the resulting humanized anti-SEMA4D antibody would comprise partially human framework regions within the variable domain of the heavy and/or light chain.

Furthermore, humanized antibodies can comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the CDRs correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al., Nature 331:522-525 (1986); Riechmann et al., Nature 332:323-329 (1988); and Presta, Curr. Op. Struct. Biol. 2:593-596 (1992); herein incorporated by reference. Accordingly, such “humanized” antibodies can include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

Antibodies that bind SEMA4D have been described the art. See, for example, US Publ. No. 2008/0219971 A1, International Patent Application WO 93/14125 and Herold et al., Int. Immunol. 7(1): 1-8 (1995), each of which is herein incorporated in its entirety by reference.

The disclosure generally relates to a method of alleviating symptoms in a subject having HD, e.g., a human patient, comprising administration of an antibody or antigen-binding fragment thereof which specifically binds to SEMA4D, or an antigen-binding fragment, variant, or derivative thereof, in combination with at least one HTT-lowering agent, as described herein. In certain embodiments, the antibody or antigen-binding fragment thereof blocks the interaction of SEMA4D with one or more of its receptors, e.g., Plexin-B 1. In certain embodiments, the antibody or antigen-binding fragment thereof specifically binds to SEMA4D and inhibits SEMA4D-mediated Plexin-B1 signal transduction. Anti-SEMA4D antibodies and antigen-binding fragments thereof having these properties can be used in the methods provided herein. Antibodies that can be used include, but are not limited to MAbs VX15/2503, MAb 67, MAb 76, D2517, D2585, and antigen-binding fragments, variants, or derivatives thereof which are fully described in U.S. Pat. No. 8,496,938 or U.S. Patent Appl. No. 2021/0032329, which are incorporated herein by reference. Additional antibodies which can be used in the methods provided herein include the BD16 and BB18 antibodies described in U.S. Pat. No. 8,067,247 as well as antigen-binding fragments, variants, or derivatives thereof; or any of MAb 301, MAb 1893, MAb 657, MAb 1807, MAb 1656, MAb 1808, Mab 59, MAb 2191, MAb 2274, MAb 2275, MAb 2276, MAb 2277, MAb 2278, MAb 2279, MAb 2280, MAb 2281, MAb 2282, MAb 2283, MAb 2284, and MAb 2285, as well as any fragments, variants or derivatives thereof as described in US 2008/0219971 A1. In certain embodiments an anti-SEMA4D antibody for use in the methods provided herein binds human, murine, or both human and murine SEMA4D. Also useful are antibodies which bind to the same epitope as any of the aforementioned antibodies and/or antibodies which competitively inhibit any of the aforementioned antibodies.

IV. Combination Therapy: Compositions and Administration Methods

Methods of preparing and administering anti-SEMA4D binding molecules, e.g., antibodies, or antigen-binding fragments, variants, or derivatives thereof to a subject in need thereof are well known to or are readily determined by those skilled in the art. The route of administration of the anti-SEMA4D binding molecule component of the disclosed combination therapy, e.g, antibody, or antigen-binding fragment, variant, or derivative thereof, can be, for example, oral, parenteral, intrathecal, Intracerebroventricular injection or by inhalation or topical administration. The term parenteral as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g., acetate, phosphate or citrate buffer), a surfactant (e.g., polysorbate), optionally a stabilizer agent (e.g., human albumin), etc. However, in other methods compatible with the teachings herein, anti-SEMA4D binding molecules, e.g., antibodies, or antigen-binding fragments, variants, or derivatives thereof can be delivered directly to the site of the adverse cellular population thereby increasing the exposure of the diseased tissue to the therapeutic agent.

Methods of preparing and administering HTT-lowering agents, such as HTT ASOs, RNAi or ribozymes or DNA enzymes, for example, to a subject in need thereof are well known to or are readily determined by those skilled in the art. (Roberts, T. et al., Advances in Oligonucleotide Drug Delivery, (2020), Nature Reviews Drug Discovery, 19:673-694; Juliano, R. L., The delivery of therapeutic oligonucleotides (2016) Nucleic Acids res., 44(14): 6518-6548). The route of administration of the HTT-lowering agent component of the combination therapy disclosed herein, e.g., an ASO or RNAi, can be by peripheral administration, local delivery, e.g., to the spinal column, although other routes of administration can be used, such as intraventricular injection. In general, an ASO is administered by intrathecal injection into the spinal column or subarachnoid space so that it reaches the cerebral spinal fluid (CSF), via intraventricular injection, or via instrastriatal injection into the corpus striatum or by injection into the cortex or any combination thereof. Also contemplated is the use of an implant or pump, such as an Ommaya reservoir, to deliver the HTT ASO (and anti-SEMA4D antibody or antigen-binding fragment thereof) directly to the fluid surrounding the brain.

Also contemplated for delivery of either or both antibody and ASO is a brain shuttle vector that facilitates penetration of large molecules such as antibodies and ASOs through the blood-brain barrier (BBB). A brain shuttle, for example, can exploit an antibody able to bind to a protein receptor located on the BBB surface exposed to peripheral blood circulation, such as the transferrin receptor, that is able to transport that antibody or antibody binding fragment together with any other linked molecule through the BBB into the brain parenchyma (Anesten, F. and Jansson, J-O. Blood-brain shuttles—a new way to reach the brain? (2021) Nature Metabolism, 3:1040-1041.

As discussed herein, anti-SEMA4D binding molecules, e.g., antibodies, or antigen-binding fragments, variants, or derivatives thereof and HTT-lowering agents, e.g., ASOs, RNAi, small molecules, and the like, can be administered in a pharmaceutically effective amount for the in vivo treatment of HD. In this regard, it will be appreciated that the disclosed SEMA4D binding molecules and HTT-lowering agents can be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an anti-SEMA4D binding molecule, e.g., an antibody, or antigen-binding fragment, variant, or derivative thereof, and an HTT-lowering agent, e.g., an ASO targeted to an HTT SNP or a non-specific HTT ASO shall be held to mean an amount sufficient to achieve effective binding to a target to achieve a benefit, e.g., improve the symptoms associated with HD such as reducing anxiety-like behavior, improving spatial memory, increasing locomotion, and any combination thereof.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and can be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences (Mack Publishing Co.) 16th ed. (1980).

In any case, sterile injectable solutions can be prepared by incorporating an active compound (e.g., at least one anti-SEMA4D antibody, or antigen-binding fragment, variant, or derivative thereof) or an HTT-lowering agent (e.g., at least one ASO targeted at an HTT SNP or a non-selective HTT ASO), each by itself or in combination in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture can have labels or package inserts indicating that the associated compositions are useful for treating a subject suffering from or predisposed to a disease or disorder.

The amount of an anti-SEMA4D binding molecule, e.g., antibody, or fragment, variant, or derivative thereof, and/or HTT-lowering agent, e.g., a non-selective ASO targeted to the HTT gene or an allele-selective ASO targeted to an HTT SNP, that is combined with the carrier materials to produce a single dosage form or separate dosage forms for each of the anti-SEMA4D binding molecule and the HTT-lowering agent will vary depending upon the host treated and the particular mode of administration. The compositions can be administered as a single dose, multiple doses or over an established period of time in an infusion. Dosage regimens also can be adjusted to provide the optimum desired response (e.g., a therapeutic or prophylactic response).

In keeping with the scope of the present disclosure, anti-SEMA4D antibodies, or antigen-binding fragments, variants, or derivatives thereof and HTT-lowering agents can be administered to a human or other animal in accordance with the aforementioned methods of treatment in an amount of each sufficient to produce a therapeutic effect. The anti-SEMA4D antibodies, or antigen-binding fragments, variants or derivatives thereof and the HTT-lowering agent can be administered to such human or other animal in a conventional dosage form prepared by combining each of the active components of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-SEMA4D binding molecules, e.g., antibodies, or antigen-binding fragments, variants, or derivatives thereof, of the disclosure can be used, as well as a cocktail of comprising one or more HTT-lowering agents, e.g., non-selective and/or selective HTT ASOs.

Therapeutically effective doses of the compositions and active components of the present disclosure, for the prevention of occurrence of decrease in the incidence of symptoms, vary depending upon many different factors, including means of administration, target site, physiological state of the patient, pathological stage of the disorder, other medications administered, and whether treatment is prophylactic or therapeutic. Treatment dosages can be titrated using routine methods known to those of skill in the art to optimize safety and efficacy.

The amount of at least one anti-SEMA4D binding molecule, e.g., antibody or binding fragment, variant, or derivative thereof, and amount of at least one HTT-lowering agent, e.g., a non-selective ASO targeted to the HTT transcript or an allele-selective ASO targeted to an HTT SNP, to be administered is readily determined by one of ordinary skill in the art without undue experimentation given the present disclosure. Factors influencing the mode of administration and the respective amount of the active components of the disclosed combination therapy include, but are not limited to, the severity of the disorder, the history of the disorder, the stage of the disorder, and the age, height, weight, health, and physical condition of the individual undergoing therapy. Similarly, the amount of each of the active components of the disclosed combination therapy to be administered will be dependent upon the mode of administration and whether the subject will undergo a single dose or multiple doses of this agent.

In one aspect, disclosed herein are HD combination therapies comprising at least one SEMA4D binding molecule (e.g., an anti-SEMA4D antibody) and at least one HTT-lowering agent (e.g., an ASO targeted to an SNP of the mutant HTT gene or non-selectively targeted to the HTT gene, for example an ASO having the sequence of SEQ ID NO: 48. In one aspect, the SEMA4D binding molecule used in the combination therapy is the SEMA4D inhibitor pepinemab, a humanized version of MAb 67, and the HTT-lowering agent is an HTT ASO, such as a non-selective ASO, e.g., an ASO having the sequence of SEQ ID NO: 48 or tominersen (SEQ ID NO: 47) (Tabrizi, B. et al., Effects of IONIS-HTTRx in patients with early Huntington's disease, results of the first HTT-lowering drug trial (CT.002) Neurology (2018)); or an allele-selective ASO targeted to an HTT SNP.

In accordance with the methods of the present disclosure, at least one anti-SEMA4D binding molecule, e.g., an antibody or antigen binding fragment, variant, or derivative thereof, is used in combination with an HTT-lowering agent as described herein, to promote a positive therapeutic response with respect to Huntington's disease. A “positive therapeutic response” with respect to HD is intended to include an improvement in the symptoms associated with HD in symptomatic subjects and is also intended to include prevention and/or improvement of symptoms in asymptomatic subjects or during early onset of HD. In particular, the methods provided herein are directed to inhibiting, preventing, reducing, alleviating, or lessening the progression of HD in a patient. Thus, for example, an improvement in HD can be characterized as an absence of some or all clinically observable symptoms, a decrease in the incidence of some or all clinically observable symptoms, or a change in some or all of the clinically observable symptoms.

Where the combined therapies comprise administration of an anti-SEMA4D binding molecule, e.g., an antibody or antigen binding fragment, variant, or derivative thereof, in combination with administration of an HTT-lowering agent, the methods of the disclosure encompass coadministration, using separate formulations or a single pharmaceutical formulation, with simultaneous or consecutive administration in either order.

It is understood and herein contemplated that while a single administration of the components of the disclosed HD combination therapies (i.e., administration of at least one SEMA4D binding molecule, such as an anti-SEMA4D antibody and at least one HTT-lowering agent) would be ideal, not every patient will respond in the same manner. ASOs do not require a viral vector for administration and do not transduce the cell; therefore, repeated administration of the ASO is preferable. In general, it is contemplated that each of the SEMA4D binding molecule, e.g., an anti-SEMA4D antibody and HTT-lowering agent, e.g., an HTT ASO will be administered to a subject to treat the symptoms of HD at least once per month or once every two, three, four, six, eight, or twelve months or other such time as needed, however, the treatment protocol will vary depending on a variety of factors such as the stage of the disease, the patient's age and health, and the like. It is further understood and herein contemplated that the order and duration of the administered components can vary as appropriate for the subject being treated.

It is intended herein that the disclosed methods of treating, inhibiting, reducing, and/or preventing symptoms of HD can be augmented with any other therapeutic treatment of HD. The additional therapy is administered prior to, during, or subsequent to administration of the anti-SEMA4D binding molecule, e.g., antibody or antigen binding fragment, variant, or derivative thereof, and HTT-lowering agent, e.g., an ASO targeted to an SNP of the HTT gene.

All of the references cited herein, are incorporated herein by reference in their entireties.

The following examples are offered by way of illustration and not by way of limitation.

### EXAMPLES

To evaluate the potential of the combination of an anti-SEMA4D immunotherapy and

HTT-lowering combination therapy for the treatment of HD, Hu97/18 HD mice were treated with a non-specific (total) HTT lowering ASO at 6 weeks of age followed by weekly MAB67 anti-SEMA4D antibody treatment until 12 months of age. Combination therapy was more effective than single agent therapy at preventing anxiety-like behavior and hypoactivity and reducing brain atrophy in striatum, corpus callosum and cortex. These findings, which are discussed below, support the use of combination anti-SEMA4D immunotherapy and HTT-lowering for the treatment of HD.

### Example 1. Experimental Design

A humanized mouse model of HD, Hu97/18, was used for these studies. Hu97/18 contain a human huntingtin gene with an expanded number of CAG repeats and exhibit evidence of HD behavior and pathology. Hu97/18 mice lack the mouse homolog (Hdh) but have one human muHTT gene and one human wtHTT gene. Hu97/18 mice were generated by intercrossing BACHD (which express full-length human muHTT with 97 CAG repeats) and YAC18 (which express full-length human wtHTT) on the Hdh−/− (which lack mouse homolog Hdh) background. Hu97/18 mice recapitulate the genetics of HD having two full-length, genomic human HTT transgenes heterozygous for the HD mutation and polymorphisms associated with HD in populations of Caucasian descent. (Southwell A L, Warby S C, Carroll J B, Doty C N, Skotte N H, Zhang W, Villanueva E B, Kovalik V, Xie Y, Pouladi M A, et al: A fully humanized transgenic mouse model of Huntington disease. Hum Mol Genet 2013, 22:18-34).

Hu 18/18 mice are humanized for the wtHTT gene (i.e., lack the mouse homolog Hdh and have a normal number of CAG repeats in both alleles) and serve as controls.

Treatment: For Examples 2-4 and 6-8 below, Hu97/18 and Hu18/18 animals were treated with an ASO having the sequence CTCAGTAACATTGACACCAC (SEQ ID NO: 48) (HH1 ASO, a non-selective HTT-lowering ASO) or PBS vehicle at 6 weeks of age, followed by weekly MAb67 or Control IgG treatment until 12 months of age. ASO or PBS vehicle was administered by single time point intracerebroventricular (ICV) injection at 6 weeks of age.

Antibodies were administered weekly at 50 mg/kg between 6 weeks and 5 months of age;

the dose was increased to 100 mg/kg between 5-12 months of age.

Behavioral assessments of open field exploration was conducted at 3 and 9 months of age. Elevated plus maze exploration was conducted at 6 months of age.

Neuropathological changes in the cortex, striatum, and corpus callosum were analyzed by immunohistochemistry in collected brains from 12 months old control and treated mice.

Relevant power calculations determined for therapeutic trials in the mice using behavioral endpoints require a minimum of 15 animals per group. The group sizes evaluated for each treatment arm are listed below:
WT Hu18/18 (n=18)
HD Hu97/18 (n=19)
HD Hu97/18+ASO (n=19)
HD Hu97/18+Mab67 (n=19)
HD Hu97/18+COMBO (n=22)

2-way ANOVA was employed to compare the effect of genotype and their interaction in the control treated groups, Hu97/18 vs Hu18/18. 1 way ANOVA was employed to compare all four treatments within the Hu97/18 genotype. In the examples, * p<0.05, ** p<0.01, *** p<0.001, **** p<0.0001.

### Example 2. Analysis of Hypoactivity of Mice During Open Field Exploration

The Open Field Exploration Test is a straight-forward test to investigate activity, anxiety-related behavior, and exploratory behavior in rodents. One parameter is thigmotaxis; the more time the animal spends hugging the walls and avoiding open space, the more anxious it probably is.

The Open Field Exploration Test is used to provide a qualitative and quantitative measurement of exploratory and locomotor activity in rodents. The apparatus consists of an arena surrounded by high walls, to prevent escape. Total distance traveled, ambulatory versus resting time, and average velocity are measures used to describe locomotor activity, which reflect exploratory behavior, while entries into and time spent in the center of the field are measures of anxiety. Measures such as total distance traveled mirror the 6 min walk test, a clinical trial outcome measure.

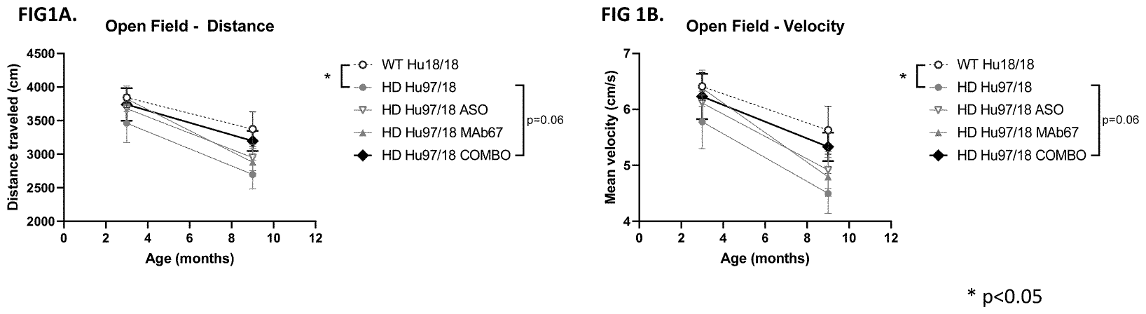

The Open Field Test was performed on individual age-matched mice (identified in Example 1) for a period of 10 minutes while being recorded by a ceiling mounted video camera. The recorded footage was analyzed by an automated tracking system to determine the average distance moved and velocity of movement for each group of mice. For open field exploration, activity was assessed by total distance traveled and mean velocity. Anxiety-like behavior was further assessed by entries into and time spent in the center of the field. The results for exploratory activity are shown in FIGS. 1A and 1B.

HD Hu97/18 mice display hypoactivity during open field exploration that is rescued by combination therapy. COMBO (combination of HH1 ASO+MAb67) treatment resulted in a strong trend toward improved behavior in HD Hu97/18 mice (p=0.06). In contrast, treatment with HH1 ASO or Mab67 single agents was not effective. FIG. 1A: open arm distance. (HH1 p=0.41; MAb67 p=0.33; COMBO p=0.06) FIG. 1B: open arm velocity (HH1 p=0.43; MAb67 p=0.34; COMBO p=0.06).

Although the difference between the Hu97/18 control and combination therapy-treated mice was not statistically significant in this test, the data demonstrate a trend toward less hypoactivity in the combination therapy-treated mice performing similarly to wild-type mice, which is not shown with either ASO or MAb alone. This trend is supported by the data in Examples 3 and 43.

### Example 3. Analysis of Anxiety-Like Behavior During Open Field Exploration

The Open Field Test was performed on individual mice (identified in Example 1) for a period of 10 minutes while being recorded by a ceiling mounted video camera. The recorded footage was analyzed by an automated tracking system to determine the average number of times each group of mice (identified in Example 1) entered the center of the field and the average amount of time each group spent in the center of the field, as an assessment of anxiety-like behavior. The results are shown in FIGS. 2A and 2B. The control treated HD Hu97/18 mice display anxiety-like behavior during the Open Field Test that is rescued by the combination therapy.

The results demonstrate that HD Hu97/18 mice display significantly more anxiety-like behavior during open field exploration, when compared to control Hu18/18 mice, which express wild-type HuHTT. COMBO (combination of HH1 ASO+MAb67) treatment significantly improved behavior in HD Hu97/18 mice. In contrast, effect with single agents was not significant (although MAb67 treatment achieved significance in center time). FIG. 2A: Center Entries. FIG. 2B: Center Time.

### Example 4. Analysis of Anxiety-Like Behavior During Elevated Plus Maze Testing

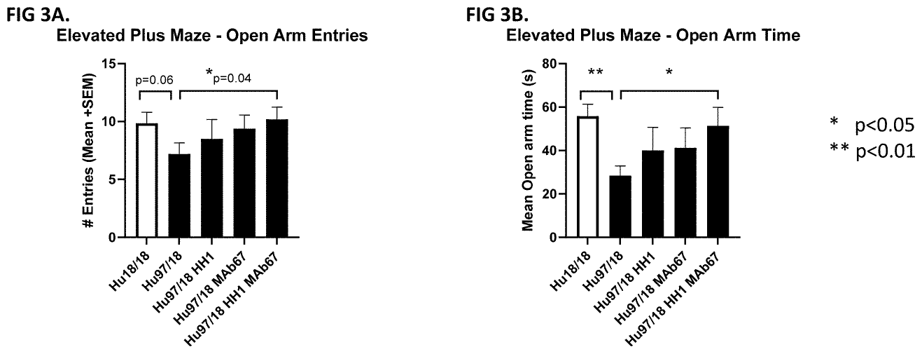

The Elevated Plus Maze (EPM) test is used to assess anxiety-related behavior in rodent models of CNS disorders. The EPM apparatus consists of a “+”-shaped maze elevated above the floor with two oppositely positioned closed arms, two oppositely positioned open arms, and a center area. Each mouse was placed in the cross section of the elevated plus maze apparatus and exploration of the maze was recorded for 5 minutes by a ceiling mounted video camera. The time spent in the open arms and the number of entries into the open arms were recorded as an assessment of anxiety. The results are shown in FIGS. 3A and 3B. The data show that HD Hu97/18 mice display anxiety-like behavior during elevated plus maze exploration that is rescued by treatment with a combination of ASO HH1 and MAb67 as described in Example 1. A statistically significant difference was observed between the behavior of the HD Hu97/18 control mice and that of the combination therapy-treated mice in terms of both entries into the open arms and time spent in the open arm area of the maze. These data also demonstrate that the combination therapy is significantly more effective than treatment with either ASO or MAb67 alone, neither of which resulted in a significant difference.

The data obtained from the Open Field Tests and Elevated Plus Maze test of Examples 2-4 demonstrate that in all instances, the combination-treated Hu97/18 mice showed significant difference in behavior (anxiety and hypoactivity) from the control treated HU97/18 mice. In contrast, single agent treated Hu97/18 mice did not always exhibit significant improvement in anxiety or hypoactivity. That is, in only some instances a single agent had a beneficial effect on anxiety or hypoactivity, but in all instances, the combination therapy had a beneficial effect on anxiety and hypoactivity.

### Example 5. Analysis of Striatal Volume Loss

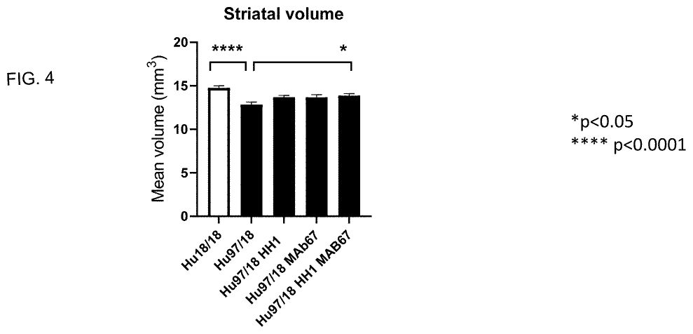

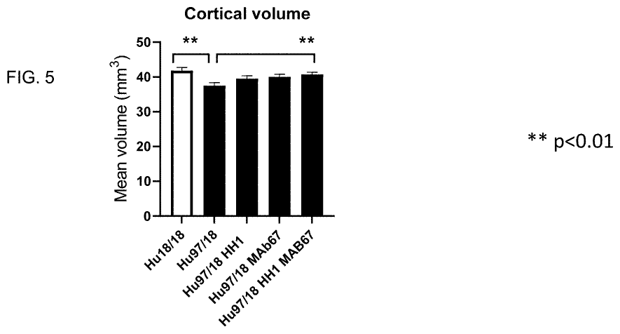

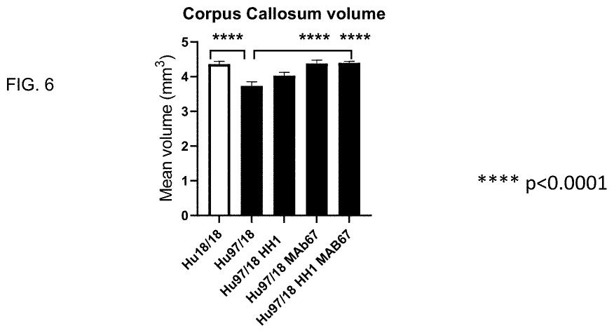

To evaluate neuropathological changes in Hu97/18 mice, striatal, cortical and corpus callosum volume were assessed in the brains of 12-month-old mice. Compared with Hu18/18 mice, Hu97/18 mice show significant reductions in striatal volume (FIG. 4, (p≤0.0001) cortex (FIG. 5, p<0.01) and corpus callosum (FIG. 6, p≤0.0001). These data are consistent with human HD in which striatal volume loss, cortical shrinkage and white matter loss are observed.

Twelve month old Hu97/18 mice treated with single agent (HH1 ASO or MAb67) or a combination of HH1 and MAb67 were also evaluated for changes in volume in the striatum (FIG. 4), cortex (FIG. 5), and corpus callosum (FIG. 6). As can be seen in FIGS. 4, 5, and 6, respectively, COMBO (combination of ASO+MAb67) therapy significantly preserved striatal, cortical, and corpus callosum volume in HD Hu97/18 mice relative to age-matched Hu97/18 control mice. In contrast, effect with single agents was significant only for the anti-SEMA4D antibody-treated mice in the corpus callosum.

### Example 6. Analysis of SEMA4D Expression in Neurons

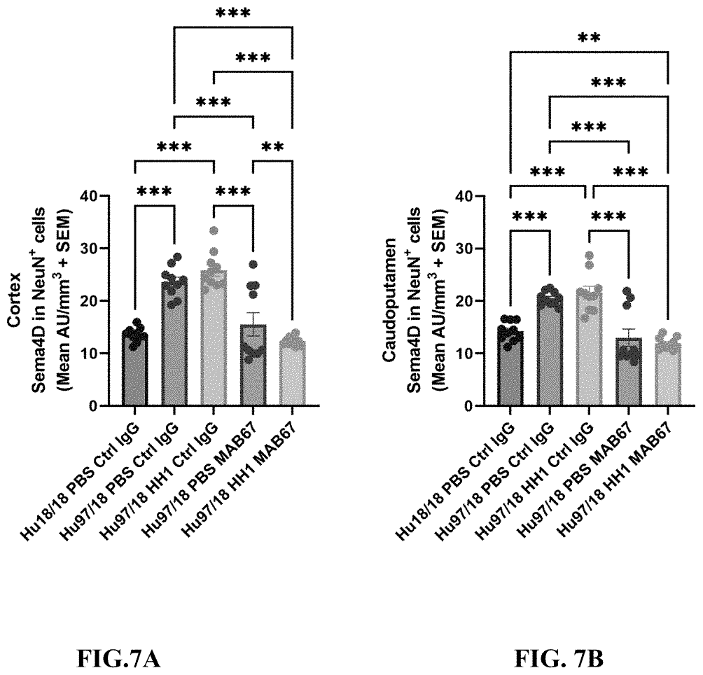

To evaluate the effect of single agent (Mab67 or HH1 ASO) and the combination (Mab67 and HH1 ASO) on expression of SEMA4D, the amount of SEMA4D in the cortex and Caudoputamen (largest part of the dorsal striatum) was assessed by immunohistochemistry in Hu18/18 and Hu97/18 mice at 12 months of age following treatment as described above. The results are shown in FIG. 7A (cortex) and 7B (Caudoputamen). As can be seen, SEMA4D is upregulated in NeuN+ neurons in HD (Hu97/18) mice, compared to Hu18/18 mice. This is consistent with data in another HD mouse model, Q175, and human brain (Evans et al. 2022, Semaphorin 4D is upregulated in neurons of diseased brains and triggers astrocyte reactivity. J Neuroinflammation 19, 200 (2022)). Treatment with anti-SEMA4D MAb67 and the combination of MAb67+ HH1 ASO, but not HH1 ASO, reduced levels of SEMA4D in Hu97/18 mice. Reduction of SEMA4D levels is significantly improved with combination therapy in the cortex.

These data demonstrate target engagement of Mab67, i.e., that the antibody binds to and specifically inhibits expression of SEMA4D.

### Example 7. Analysis of Effect of Treatment With Hill ASO on Plasma HTT Level

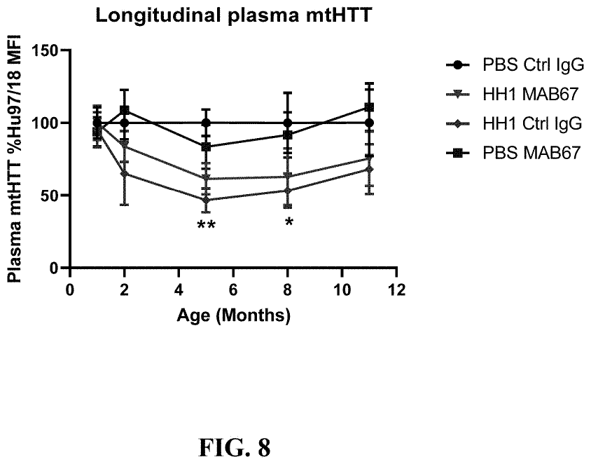

To evaluate the effect of single agent HH1 ASO on the level of mutant HTT protein (mtHTT) in plasma, the amount of mtHTT was assessed in Hu97/18 mice at 12 months of age following treatment as described above (Example 1, Experimental Design). The results are shown in FIG. 8. As can be seen, treatment with HH1 ASO reduced levels of mutant HTT protein (mtHTT) in plasma. In contrast, treatment with MAb67 single agent does not significantly impact levels of mtHTT.

These data suggest target engagement of HH1, i.e., HH1 ASO specifically inhibits mtHTT.

### Example 8. Analysis of Treatment Effect on Reactive Astrocytes

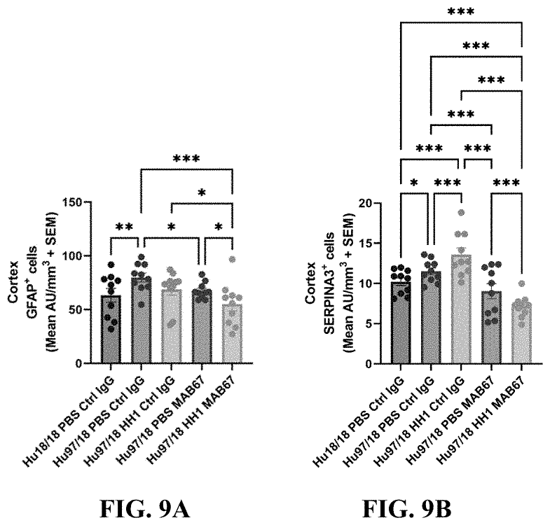

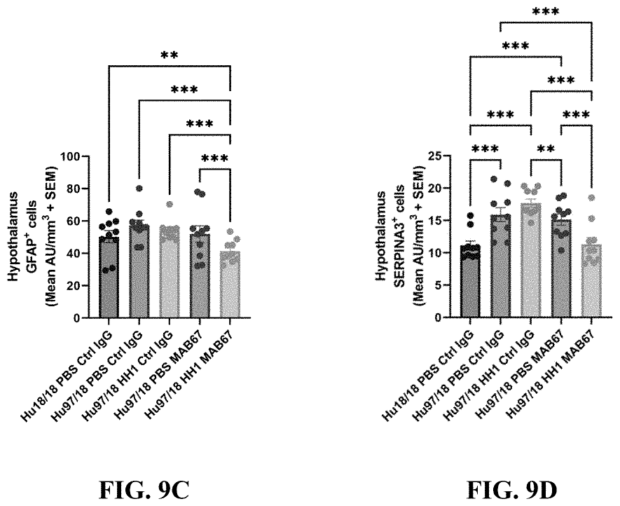

To determine the effect of single agent and combination treatment on reactive astrocytes in the cortex and hypothalamus in HD (Hu97/18) mice, mice were treated as above and reactive astrocytes were detected by immunohistochemical staining (mean fluorescence intensity (MFI)) for glial fibrillary acidic protein (GFAP) and SerpinA3, which are biomarkers of reactive astrocytes. (See Escartin C, et al., Reactive astrocyte nomenclature, definitions, and future directions. Nat Neurosci. 2021 March; 24(3):312-325. doi: 10.1038/s41593-020-00783-4. Epub 2021 Feb. 15. PMID: 33589835; PMCID: PMC8007081; and Viejo L, Noori A, Merrill E, Das S, Hyman B T, Serrano-Pozo A. Systematic review of human post-mortem immunohistochemical studies and bioinformatics analyses unveil the complexity of astrocyte reaction in Alzheimer's disease. Neuropathol Appl Neurobiol. 2022 February; 48(1):e12753. doi: 10.1111/nan.12753. Epub 2021 Aug. 17. PMID: 34297416; PMCID: PMC8766893, incorporated herein in their entirety). The results are shown in FIGS. 9A-D. As can be seen, the number of reactive astrocytes is significantly increased in Hu97/18 HD mice compared to Hu18/18 mice. The combination of MAb67+HH1 ASO significantly improved reduction of GFAP+ and SERPINA3+ reactive astrocytes in the cortex (FIGS. 9A and B, respectively) and hypothalamus (FIGS. 9C and D, respectively) of Hu97/18 mice, compared to control and to single agents. * p<0.05; ** p<0.01, *** p<0.001, **** p<0.0001.

Sequences

SEQ ID NO: 1: Semaphorin-4D Homo sapiens: Met Arg Met Cys Thr Pro Ile Arg Gly Leu Leu
Met Ala Leu Ala Val Met Phe Gly Thr Ala Met Ala Phe Ala Pro Ile Pro Arg Ile Thr Trp Glu His
Arg Glu Val His Leu Val Gln Phe His Glu Pro Asp Ile Tyr Asn Tyr Ser Ala Leu Leu Leu Ser Glu
Asp Lys Asp Thr Leu Tyr Ile Gly Ala Arg Glu Ala Val Phe Ala Val Asn Ala Leu Asn Ile Ser
GluLys Gln His Glu Val Tyr Trp Lys Val Ser Glu Asp Lys Lys Ala Lys Cys Ala Glu Lys Gly Lys
Ser Lys Gln Thr Glu Cys Leu Asn Tyr Ile 100 105 110Arg Val Leu Gln Pro Leu Ser Ala Thr Ser
Leu Tyr Val Cys Gly Thr Asn Ala Phe Gln Pro Ala Cys Asp His Leu Asn Leu Thr Ser Phe Lys
Phe Leu Gly Lys Asn Glu Asp Gly Lys Gly Arg Cys Pro Phe Asp ProAla His Ser Tyr Thr Ser Val
Met Val Asp Gly Glu Leu Tyr Ser Gly Thr Ser Tyr Asn Phe Leu Gly Ser Glu Pro Ile Ile Ser Arg
Asn Ser Ser His Ser Pro Leu Arg Thr Glu Tyr Ala Ile Pro Trp Leu Asn Glu Pro Ser Phe Val Phe
Ala Asp Val Ile Arg Lys Ser Pro Asp Ser Pro Asp Gly Glu Asp Asp Arg Val Tyr Phe Phe Phe Thr
Glu Val Ser ValGlu Tyr Glu Phe Val Phe Arg Val Leu Ile Pro Arg Ile Ala Arg Val Cys Lys Gly
Asp Gln Gly Gly Leu Arg Thr Leu Gln Lys Lys Trp Thr Ser Phe Leu Lys Ala Arg Leu Ile Cys Ser
Arg Pro Asp Ser Gly Leu Val Phe Asn Val Leu Arg Asp Val Phe Val Leu Arg Ser Pro Gly Leu
Lys Val Pro Val Phe Tyr Ala Leu Phe Thr Pro Gln Leu Asn Asn Val305 Gly Leu Ser Ala Val Cys
Ala Tyr Asn Leu Ser Thr Ala Glu Glu ValPhe Ser His Gly Lys Tyr Met Gln Ser Thr Thr Val Glu
Gln Ser His hr Lys Trp Val Arg Tyr Asn Gly Pro Val Pro Lys Pro Arg Pro Gly la Cys Ile Asp Ser
Glu Ala Arg Ala Ala Asn Tyr Thr Ser Ser Leu Asn Leu Pro Asp Lys Thr Leu Gln Phe Val Lys
Asp His Pro Leu MetAsp Asp Ser Val Thr Pro Ile Asp Asn Arg Pro Arg Leu Ile Lys Lys Asp Val
Asn Tyr Thr Gln Ile Val Val Asp Arg Thr Gln Ala Leu Asp Gly Thr Val Tyr Asp Val Met Phe
Val Ser Thr Asp Arg Gly Ala Leu His Lys Ala Ile Ser Leu Glu His Ala Val His Ile Ile Glu Glu
Thr Gln Leu Phe Gln Asp Phe Glu Pro Val Gln Thr Leu Leu Leu Ser Ser Lys Lys Gly Asn Arg
Phe Val Tyr Ala Gly Ser Asn Ser Gly Val Val Gln Ala Pro Leu Ala Phe Cys Gly Lys His Gly Thr
Cys Glu Asp CysVal Leu Ala Arg Asp Pro Tyr Cys Ala Trp Ser Pro Pro Thr Ala Thr Cys Val Ala
Leu His Gln Thr Glu Ser Pro Ser Arg Gly Leu Ile GlnGlu Met Ser Gly Asp Ala Ser Val Cys Pro
Asp Lys Ser Lys Gly SerTyr Arg Gln His Phe Phe Lys His Gly Gly Thr Ala Glu Leu Lys Cys Ser
Gln Lys Ser Asn Leu Ala Arg Val Phe Trp Lys Phe Gln Asn Gly Val Leu Lys Ala Glu Ser Pro
Lys Tyr Gly Leu Met Gly Arg Lys Asn 595 600 605Leu Leu Ile Phe Asn Leu Ser Glu Gly Asp
Ser Gly Val Tyr Gln Cys Leu Ser Glu Glu Arg Val Lys Asn Lys Thr Val Phe Gln Val Val AlaLys
His Val Leu Glu Val Lys Val Val Pro Lys Pro Val Val Ala ProThr Leu Ser Val Val Gln Thr Glu
Gly Ser Arg Ile Ala Thr Lys ValLeu Val Ala Ser Thr Gln Gly Ser Ser Pro Pro Thr Pro Ala Val
Gln Ala Thr Ser Ser Gly Ala Ile Thr Leu Pro Pro Lys Pro Ala Pro Thr Gly Thr Ser Cys Glu Pro
Lys Ile Val Ile Asn Thr Val Pro Gln Leu His Ser Glu Lys Thr Met Tyr Leu Lys Ser Ser Asp Asn
Arg Leu Leu Met Ser Leu Phe Leu Phe Phe Phe Val Leu Phe Leu Cys Leu Phe Phe Tyr Asn Cys
Tyr Lys Gly Tyr Leu Pro Arg Gln Cys Leu Lys Phe Arg Ser Ala Leu Leu Ile Gly Lys Lys Lys Pro
Lys Ser Asp Phe Cys Asp Arg Glu Gln Ser Leu Lys Glu Thr Leu Val Glu Pro Gly Ser Phe SerGln
Gln Asn Gly Glu His Pro Lys Pro Ala Leu Asp Thr Gly Tyr Glu Thr Glu Gln Asp Thr Ile Thr Ser
Lys Val Pro Thr Asp Arg Glu Asp Ser Gln Arg Ile Asp Asp Leu Ser Ala Arg Asp Lys Pro Phe
Asp Val Lys Cys Glu Leu Lys Phe Ala Asp Ser Asp Ala Asp Gly Asp
SEQ ID NO: 2: Semaphorin-4D Murine: Met Arg Met Cys Ala Pro Val Arg Gly Leu Phe Leu
Ala Leu Val Val1 Val Leu Arg Thr Ala Val Ala Phe Ala Pro Val Pro Arg Leu Thr Trp Glu His
Gly Glu Val Gly Leu Val Gln Phe His Lys Pro Gly Ile Phe Asn Tyr Ser Ala Leu Leu Met Ser Glu
Asp Lys Asp Thr Leu Tyr Val Gly Ala Arg Glu Ala Val Phe Ala Val Asn Ala Leu Asn Ile Ser
GluLys Gln His Glu Val Tyr Trp Lys Val Ser Glu Asp Lys Lys Ser LysCys Ala Glu Lys Gly Lys
Ser Lys Gln Thr Glu Cys Leu Asn Tyr Ile Arg Val Leu Gln Pro Leu Ser Ser Thr Ser Leu Tyr Val
Cys Gly Thr Asn Ala Phe Gln Pro Thr Cys Asp His Leu Asn Leu Thr Ser Phe Lys Phe Leu Gly
Lys Ser Glu Asp Gly Lys Gly Arg Cys Pro Phe Asp Pro Ala His Ser Tyr Thr Ser Val Met Val Gly
Gly Glu Leu Tyr Ser Gly Thr Ser Tyr Asn Phe Leu Gly Ser Glu Pro Ile Ile Ser Arg Asn Ser Ser
His Ser Pro Leu Arg Thr Glu Tyr Ala Ile Pro Trp Leu Asn Glu Pro Ser Phe Val Phe Ala Asp Val
Ile Gln Lys Ser Pro Asp Gly Pro Glu Gly Glu Asp Asp Lys Val Tyr Phe Phe Phe Thr Glu Val Ser
ValGlu Tyr Glu Phe Val Phe Lys Leu Met Ile Pro Arg Val Ala Arg Val Cys Lys Gly Asp Gln Gly
Gly Leu Arg Thr Leu Gln Lys Lys Trp Thr Ser Phe Leu Lys Ala Arg Leu Ile Cys Ser Lys Pro Asp
Ser Gly Leu Val Phe Asn Ile Leu Gln Asp Val Phe Val Leu Arg Ala Pro Gly Leu Lys Glu Pro Val
Phe Tyr Ala Val Phe Thr Pro GIn Leu Asn Asn ValGly Leu Ser Ala Val Cys Ala Tyr Thr Leu Ala
Thr Val Glu Ala Val Phe Ser Arg Gly Lys Tyr Met Gln Ser Ala Thr Val Glu Gln Ser His Thr Lys
Trp Val Arg Tyr Asn Gly Pro Val Pro Thr Pro Arg Pro Gly Ala Cys Ile Asp Ser Glu Ala Arg Ala
Ala Asn Tyr Thr Ser Ser Leu Asn Leu Pro Asp Lys Thr Leu Gln Phe Val Lys Asp His Pro Leu
MetAsp Asp Ser Val Thr Pro Ile Asp Asn Arg Pro Lys Leu Ile Lys Lys Asp Val Asn Tyr Thr Gln
Ile Val Val Asp Arg Thr Gln Ala Leu Asp Gly Thr Phe Tyr Asp Val Met Phe Ile Ser Thr Asp Arg
Gly Ala LeuHis Lys Ala Val Ile Leu Thr Lys Glu Val His Val Ile Glu Glu ThrGln Leu Phe Arg
Asp Ser Glu Pro Val Leu Thr Leu Leu Leu Ser SerLys Lys Gly Arg Lys Phe Val Tyr Ala Gly Ser
Asn Ser Gly Val ValGln Ala Pro Leu Ala Phe Cys Glu Lys His Gly Ser Cys Glu Asp Cys Val Leu
Ala Arg Asp Pro Tyr Cys Ala Trp Ser Pro Ala Ile Lys Ala Cys Val Thr Leu His Gln Glu Glu Ala
Ser Ser Arg Gly Trp Ile Gln Asp Met Ser Gly Asp Thr Ser Ser Cys Leu Asp Lys Ser Lys Glu
SerPhe Asn Gln His Phe Phe Lys His Gly Gly Thr Ala Glu Leu Lys Cys Phe Gln Lys Ser Asn Leu
Ala Arg Val Val Trp Lys Phe Gln Asn Gly Glu Leu Lys Ala Ala Ser Pro Lys Tyr Gly Phe Val Gly
Arg Lys His Leu Leu Ile Phe Asn Leu Ser Asp Gly Asp Ser Gly Val Tyr Gln Cys Leu Ser Glu Glu
Arg Val Arg Asn Lys Thr Val Ser Gln Leu Leu Alays His Val Leu Glu Val Lys Met Val Pro Arg
Thr Pro Pro Ser Pro Thr Ser Glu Asp Ala Gln Thr Glu Gly Ser Lys Ile Thr Ser Lys Met Pro Val
Ala Ser Thr Gln Gly Ser Ser Pro Pro Thr Pro Ala Leu Trp Ala Thr Ser Pro Arg Ala Ala Thr Leu
Pro Pro Lys Ser Ser Ser Gly Thr Ser Cys Glu Pro Lys Met Val Ile Asn Thr Val Pro Gln Leu His
720Ser Glu Lys Thr Val Tyr Leu Lys Ser Ser Asp Asn Arg Leu Leu Met Ser Leu Leu Leu Phe Ile
Phe Val Leu Phe Leu Cys Leu Phe Ser Tyr Asn Cys Tyr Lys Gly Tyr Leu Pro Gly Gln Cys Leu
Lys Phe Arg Ser Ala Leu Leu Leu Gly Lys Lys Thr Pro Lys Ser Asp Phe Ser Asp Leu Glu Gln
Ser Val Lys Glu Thr Leu Val Glu Pro Gly Ser Phe Ser Gln 800Gln Asn Gly Asp His Pro Lys Pro
Ala Leu Asp Thr Gly Tyr Glu ThrGlu Gln Asp Thr Ile Thr Ser Lys Val Pro Thr Asp Arg Glu Asp
Ser Gln Arg Ile Asp Glu Leu Ser Ala Arg Asp Lys Pro Phe Asp Val Lys Cys Glu Leu Lys Phe Ala
Asp Ser Asp Ala Asp Gly Asp
SEQ ID NO: 3: Polynucleotide anti-SEMA4D VH CDR1: ggctacagct tcagcgacta ctacatgcac
SEQ ID NO: 4: Polynucleotide anti-SEMA4D VH CDR2: cagattaatc ctaccactgg cggcgctage
tacaaccaga agttcaaggg c
SEQ ID NO: 5: Polynucleotide anti-SEMA4D VH CDR3: tattactacg gcagacactt cgatgto
SEQ ID NO: 6: Polypeptide anti-SEMA4D VH CDR1: Gly Tyr Ser Phe Ser Asp Tyr Tyr Met
His
SEQ ID NO: 7: Polypeptide anti-SEMA4D VH CDR2: Gln Ile Asn Pro Thr Thr Gly Gly Ala
Ser Tyr Asn Gln Lys Phe Lys Gly
SEQ ID NO: 8: Polypeptide anti-SEMA4D VH CDR3: Tyr Tyr Tyr Gly Arg His Phe Asp Val
SEQ ID NO: 9: Polypeptide anti-SEMA4D VH 2503: Gln Val Gln Leu Val Gln Ser Gly Ala
Glu Val Lys Lys Pro Gly Ser Ser Val Lys Val Ser Cys Lys Ala Ser Gly Tyr Ser Phe Ser Asp Tyr
Tyr Met His Trp Val Arg Gln Ala Pro Gly Gln Gly Leu Glu Trp Met Gly Gln Ile Asn Pro Thr Thr
Gly Gly Ala Ser Tyr Asn Gln Lys Phe Lys Gly Lys Ala Thr Ile Thr Val Asp Lys Ser Thr Ser Thr
Ala Tyr Met Glu Leu Ser Ser Leu Arg Ser Glu Asp Thr Ala Val Tyr Tyr Cys Ala Arg Tyr Tyr Tyr
Gly Arg His Phe Asp Val Trp Gly Gln Gly Thr Thr Val Thr Val Ser Ser
SEQ ID NO: 10: Polypeptide anti-SEMA4D VH 67: Gln Val Gln Leu Gln Gln Ser Gly Pro Glu
Leu Val Lys Pro Gly Ala Ser Val Lys Ile Ser Cys Lys Ala Ser Gly Tyr Ser Phe Ser Asp Tyr Tyr
Met His Trp Val Lys Gln Ser Pro Glu Asn Ser Leu Glu Trp Ile Gly Gln Ile Asn Pro Thr Thr Gly
Gly Ala Ser Tyr Asn Gln Lys Phe Lys Gly Lys Ala Thr Leu Thr Val Asp Lys Ser Ser Ser Thr Ala
Tyr Met Gln Leu Lys Ser Leu Thr Ser Glu Glu Ser Ala Val Tyr Tyr Cys Thr Arg Tyr Tyr Tyr Gly
Arg His Phe Asp Val Trp Gly Gln Gly Thr Thr Val Thr Val Ser Ser
SEQ ID NO: 11: Polynucleotide anti-SEMA4D VL CDR1: aaggccagcc aaagcgtgga ttatgatgge
gatagctata tgaa
SEQ ID NO: 12: Polynucleotide anti-SEMA4D VL CDR2: gctgcatcca atctggaaag c
SEQ ID NO: 13: Polynucleotide anti-SEMA4D VL CDR3: cagcaaagca atgaggatcc ctacacc
SEQ ID NO: 14: Polypeptide snit-SEMA4D VL CDR1: Lys Ala Ser Gln Ser Val Asp Tyr Asp
Gly Asp Ser Tyr Met Asn
SEQ ID NO: 15: Polypeptide anti-SEMA4D VL CDR2: Ala Ala Ser Asn Leu Glu Ser
SEQ ID NO: 16: Polypeptide anti-SEMA4D VL CDR3: Gln Gln Ser Asn Glu Asp Pro Tyr
Thr
SEQ ID NO: 17: Polypeptide anti-SEMA4D 2503 VL: Asp Ile Val Met Thr Gln Ser Pro Asp
Ser Leu Ala Val Ser Leu Gly Glu Arg Ala Thr Ile Asn Cys Lys Ala Ser Gln Ser Val Asp Tyr Asp
Gly Asp Ser Tyr Met Asn Trp Tyr Gln Gln Lys Pro Gly Gln Pro Pro Lys Leu Leu Ile Tyr Ala Ala
Ser Asn Leu Glu Ser Gly Val Pro Asp Arg Phe Ser Gly Ser Gly Ser Gly Thr Asp Phe Thr Leu Thr
Ile Ser Ser Leu Gln Ala Glu Asp Val Ala Val Tyr Tyr Cys Gln Gln Ser Asn Glu Asp Pro Tyr Thr
Phe Gly Gln Gly Thr Lys Leu Glu Ile Lys
SEQ ID NO: 18: Polypeptide anti-SEMA4D 67 VL: Asp Ile Val Met Thr Gln Ser Pro Ala Ser
Leu Ala Val Ser Leu Gly Gln Arg Ala Thr Ile Ser Cys Lys Ala Ser Gln Ser Val Asp Tyr Asp Gly
Asp Ser Tyr Met Asn Trp Tyr Gln Gln Lys Pro Gly Gln Pro Pro Lys Leu Leu Ile Tyr Ala Ala Ser
Asn Leu Glu Ser Gly Ile Pro Ala Arg Phe Ser Gly Ser Gly Ser Gly Thr Asp Phe Thr Leu Asn Ile
His Pro Val Glu Glu Glu Asp Ala Ala Thr Tyr Tyr Cys Gln Gln Ser Asn Glu Asp Pro Tyr Thr Phe
Gly Gly Gly Thr Lys Leu Glu Ile Lys
SEQ ID NO: 19: Polynucleotide anti-SEMA4D VH 2503: caggtgcage tggtgcagag cggcgctgag
gtgaagaagc ctggcagcag cgtgaaggtc tcctgcaagg ctagcggcta cagcttcagc gactactaca tgcactgggt
gagacaggcc cctggccaag gcctggagtg gatgggccag attaatccta ccactggcgg cgctagctac aaccagaagt
tcaagggcaa ggccaccatt accgtggaca aaagcaccag cacagcctac atggagctga gcagcctgag aagcgaggac
accgccgtgt attactgtgc cagatattac tacggcagac acttcgatgt ctggggccaa ggcaccacgg tcaccgtctc ttca
SEQ ID NO: 20: Polynucleotide anti-SEMA4D VH 67: caggtccage tgcagcagtc tggacctgag
ctggtgaagc ctggggcttc agtgaagata tcctgcaagg cttctggtta ctcattcagt gactactaca tgcactgggt
gaagcaaagt
cctgaaaata gtcttgagtg gattggacag attaatccta ccactggggg tgctagctac aaccagaagt tcaagggcaa
ggccacatta actgtagata aatcctccag cacagcctac atgcagctca agagcctgac atctgaagag tctgcagtet
attactgtac aagatattac tacggtagac acttegatgt ctggggccaa gggaccacgg tcaccgtttc ctca
SEQ ID NO: 21: Polynucleotide anti-SEMA4D VL 2503: gacatcgtga tgacccagag cccagacagc
ctggctgtga gcctgggcga gagggccacc atcaactgca aggccagcca aagcgtggat tatgatggcg atagctatat
gaactggtac cagcagaaac caggccagcc tcctaagctg ctgatttacg ctgcatccaa tctggaaagc ggcgtgcctg
acagattcag cggcagcggc agcggcacag atttcactct gaccatcagc agcctgcagg ctgaagatgt ggcagtgtat
tactgtcagc aaagcaatga ggatccctac accttcggcc aagggaccaa gctcgagatc aaa
SEQ ID NO: 22: Polynucleotide anti-SEMA4D VL 67: gacattgtga tgacccagtc tccagcttct
ttggctgtgt ctctagggca gagggccacc atctcctgca aggccagcca aagtgttgat tatgatggtg atagttatat
gaactggtac
caacagaaac caggacagcc acccaaactc ctcatctatg ctgcatccaa tctagaatct gggatcccag ccaggtttag
tggcagtggg tctgggacag acttcaccct caacatccat cctgtggagg aggaggatgc tgcaacctat tactgtcagc
aaagtaatga ggatccgtac acgttcggag gggggaccaa gctcgagate aaa
SEQ ID NO: 23: Polypeptide anti-SEMA4D VH 76: Gln Val Gln Leu Gln Gln Ser Gly Ala Glu
Leu Ala Lys Pro Gly Ala Ser Val Lys Met Ser Cys Lys Ala Ser Gly Tyr Thr Phe Thr Arg Tyr Trp
Met His Trp Val Lys Gln Arg Pro Gly Gln Gly Leu Glu Trp Ile Gly Tyr Ile Asn Pro Ser Thr Gly
Tyr Ser Asp Tyr Asn Gln Lys Phe Lys Asp Lys Ala Thr Leu Thr Ala Asp Lys Ser Ser Ser Thr Ala
Tyr Met Gln Leu Ser Ser Leu Thr Ser Glu Asp Ser Ala Val Tyr Tyr Cys Ala Arg Asp Pro Tyr Gly
Trp Thr Met Asp Ser Trp Gly Gln Gly Thr Leu Val Thr Val Ser Ser
SEQ ID NO: 24: Polypeptide anti-SEMA4D VH 76 CDR1: Gly Tyr Thr Phe Thr Arg Tyr Trp
Met His
SEQ ID NO: 25: Polypeptide anti-SEMA4D VH 76 CDR2: Tyr Ile Asn Pro Ser Thr Gly Tyr
Ser Asp Tyr Asn Gln Lys Phe Lys Asp
SEQ ID NO: 26: Polypeptide anti-SEMA4D VH 76 CDR3: Asp Pro Tyr Gly Trp Thr Met Asp
Ser
SEQ ID NO: 27: Polypeptide anti-SEMA4D VL 76: Asp Ile Gln Met Thr Gln Ser Pro Ser Ser
Leu Ser Ala Ser Leu Gly Asp Thr Ile Thr Ile Thr Cys His Ala Ser Gln Asn Ile Asn Val Trp Leu
Ser Trp Tyr Gln Gln Lys Pro Gly Asn Ile Pro Lys Leu Leu Ile Tyr Lys Ala Ser Asn Leu His Thr
Gly Val Pro Ser Arg Phe Ser Gly Ser Gly Ser Gly Thr Gly Phe Thr Leu Thr Ile Ser Ser Leu Gln
Pro Glu Asp Ile Ala Thr Tyr Tyr Cys Gln Gln Gly Gln Ser Tyr Pro Tyr Thr Phe Gly Gly Gly Thr
Lys Leu Glu Ile Lys
SEQ ID NO: 28: Polypeptide anti-SEMA4D VL 76 CDR1: His Ala Ser Gln Asn Ile Asn Val
Trp Leu Ser
SEQ ID NO: 29: Polypeptide anti-SEMA4D VL 76 CDR2: Lys Ala Ser Asn Leu His Thr
SEQ ID NO: 30: Polypeptide anti-SEMA4D VL 76 CDR3: Gln Gln Gly Gln Ser Tyr Pro Tyr
Thr
SEQ ID NO: 31: Polynucleotide anti-SEMA4D VH 76: caggtccagc tgcagcagtc tggggctgaa
ctggcaaaac ctggggcctc agtgaagatg tcctgcaagg cttctggcta cacctttact aggtactgga tgcactgggt
aaaacagagg cctggacagg gtctggaatg gattggatac attaatccta gcactggtta ttctgattac aatcagaagt
tcaaggacaa ggccacattg actgcagaca aatcctccag cacagcctac atgcaactga gcagcctgac atctgaggac
tctgcagtct attactgtgc aagagacccc tacggctgga ctatggactc ctggggccaa gggactctgg tcaccgtctc ctca
SEQ ID NO: 32: Polynucleotide anti-SEMA4D VH 76 CDR1: ggctacacct ttactaggta ctggatgcac
SEQ ID NO: 33: Polynucleotide anti-SEMA4D VH 76 CDR2: tacattaatc ctagcactgg ttattctgat
tacaatcaga agttcaagga c
SEQ ID NO: 34: Polynucleotide anti-SEMA4D VH 76 CDR2: gacccctacg gctggactat ggactcc
SEQ ID NO: 35: Polynucleotide anti-SEMA4D VL 76: gacatccaga tgacccagtc tccatccagt
ctgtctgcat cccttggaga cacaattacc atcacttgcc atgccagtca gaacattaat gtttggttaa gctggtacca
gcagaaacca
ggaaatattc ctaaactatt gatctataag gcttccaact tgcacacagg cgtcccatca aggtttagtg gcagtggatc
tggaacaggt
ttcacattaa ccatcagcag cctgcagcct gaagacattg ccacttacta ctgtcaacag ggtcaaagtt atccgtacac
gttcggaggg gggaccaagc tcgagatcaa a
SEQ ID NO: 36: Polynucleotide anti-SEMA4D VL 76 CDR1: catgccagtc agaacattaa tgtttggtta
agc
SEQ ID NO: 37: Polynucleotide anti-SEMA4D VL 76 CDR2: aaggcttcca acttgcacac a
SEQ ID NO: 38: Polynucleotide anti-SEMA4D VL 76 CDR2: caacagggtc aaagttatcc gtacacg
SEQ ID NO: 39: Polypeptide anti-SEMA4D VH D2517: Glu Val Gln Leu Val Glu Ser Gly Gly
Gly Leu Val Gln Pro Gly GlySer Leu Arg Leu Ser Cys Ala Ala Ser Gly Phe Ile Phe Ser Asp Tyr
Trp Met Val Trp Val Arg Gln Ala Pro Gly Lys Gly Leu Glu Tyr Val Ala His Met Asn Gln Asp
Gly Gly Ala Arg Tyr Tyr Ala Glu Ser Val Arg Gly Arg Phe Thr Ile Ser Arg Asp Asn Ala Lys Asn
Ser Leu Tyr Leu Gln Met Asn Ser Leu Arg Ala Glu Asp Thr Ala Val Tyr Tyr Cys Ala Arg Asp
Pro Trp Gly Tyr Asp Ser Trp Gly Gln Gly Thr Leu Val Thr
SEQ ID NO: 40: Polypeptide anti-SEMA4D D2517 VH CDR1: Asp Tyr Trp Met Val
SEQ ID NO: 41: Polypeptide anti-SEMA4D D2517 VH CDR2: His Met Asn Gln Asp Gly Gly
Ala Arg Tyr Tyr
SEQ ID NO: 42: Polypeptide anti-SEMA4D D2517 VH CDR3: Asp Pro Trp Gly Tyr
SEQ ID NO: 43: Polypeptide anti-SEMA4D VL D2517: Ser Tyr Glu Leu Thr Gln Pro Pro Ser
Val Ser Val Ser Pro Gly GlnThr Ala Ser Ile Thr Cys Ser Gly Asp Lys Leu Gly Asp Lys Tyr Ala
Val Trp Tyr Gln Gln Lys Pro Gly Gln Ser Pro Val Leu Val Ile TyrGln Asp Ser Lys Arg Pro Ser
Gly Ile Pro Glu Arg Phe Ser Gly SerAsn Ser Gly Asn Thr Ala Thr Leu Thr Ile Ser Gly Thr Gln
Ala Met0Asp Glu Ala Asp Tyr Tyr Cys Gln Ala Trp Glu Gln Glu Ala Ala TrpVal Phe Gly Gly
Gly Thr Lys Leu
SEQ ID NO: 44: Polypeptide anti-SEMA4D D2517 VL CDR1: Ser Gly Asp Lys Leu Gly Asp
Lys Tyr Ala Val
SEQ ID NO: 45: Polypeptide anti-SEMA4D D2517 VL CDR2: Gln Asp Ser Lys Arg Pro Ser
SEQ ID NO: 46: Polypeptide anti-SEMA4D D2517 VL CDR1: Gln Ala Trp Glu Gln Glu Ala
Ala Trp Val
SEQ ID NO: 47: antisense oligonucleotide tominersen: ctₒcₒaₒgTAACATTGACaₒcₒcₒac,
wherein capital letters represent 2′-deoxyribose nucleosides, and small letters represent 2′-
(2-methoxyethyl) ribose nucleosides; o represents a phosphodiester linkage and all other
linkages are phosphorothioate; letters “a” and “A” represent adenine, “c” and “C” represent
5-methylcytosine, “g” and “G” represent guanine, and “t” and “T” represent thymine nucleobases
SEQ ID NO: 48: antisense oligonucleotide HH1: CTCAGTAACATTGACACCAC

1. A combination therapy for the treatment of Huntington's disease (HD) comprising at least one isolated antibody or antigen-binding fragment thereof that specifically binds to semaphorin-4D (SEMA4D) and a therapeutically effective amount of at least one HTT-lowering agent.

2. The combination therapy of claim 1, wherein the antibody or antigen-binding fragment thereof inhibits SEMA4D interactions with its receptor.

3. The combination therapy of claim 2, wherein the receptor is Plexin-B1.

4. The combination therapy of claim 1, wherein the antibody or antigen-binding fragment thereof inhibits SEMA4D-mediated Plexin-B1 signal transduction.

5. The combination therapy of claim 1, wherein the antibody or antigen-binding fragment thereof competitively inhibits a reference monoclonal antibody VX15/2503 or MAb67 from specifically binding to SEMA4D.

6. The combination therapy of claim 1, wherein the antibody or antigen-binding fragment thereof comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively.

7. The combination therapy of claim 6, wherein the VH and VL comprise, respectively, SEQ ID NO: 9 and SEQ ID NO: 17 or SEQ ID NO: 10 and SEQ ID NO: 18.

8. The combination therapy of claim 1, wherein the at least one huntingtin (HTT)-lowering agent is an antisense oligonucleotide (ASO).

9. The combination therapy of claim 8, wherein the ASO is an allele-selective ASO or a non-selective ASO.

10. The combination therapy of claim 9, wherein the ASO is a non-selective ASO.

11. The combination therapy of claim 1, wherein the ASO and antibody or antigen-binding fragment thereof are administered separately or concurrently.

12. The combination therapy of claim 11, wherein administration of the combination of the isolated antibody or antigen-binding fragment thereof and the HTT-lowering agent results in enhanced therapeutic efficacy relative to administration of either the isolated antibody or antigen-binding fragment thereof or the HTT-lowering agent alone.

13. The combination of claim 1, wherein administration of the combination of the isolated antibody or antigen-binding fragment thereof and the HTT-lowering agent results in improvement of neuropsychiatric symptoms, cognitive symptoms, motor dysfunction, brain atrophy, metabolic activity, or any combination thereof.

14. The combination therapy of claim 13, wherein the improvement of neuropsychiatric symptoms is selected from the group consisting of reduced anxiety-like behavior, improved spatial memory, increased locomotion, and any combination thereof.

15. A method of treating a subject having Huntington's disease (HD) with a combination therapy comprising administering at least one isolated antibody or antigen-binding fragment thereof that specifically binds to semaphorin-4D (SEMA4D) and a therapeutically effective amount of at least one HTT-lowering agent.

16. The method of claim 15, wherein the antibody or antigen-binding fragment thereof inhibits SEMA4D interactions with its receptor.

17. The method of claim 16, wherein the receptor is Plexin-B1.

18. The method of claim 17, wherein the antibody or antigen-binding fragment thereof inhibits SEMA4D-mediated Plexin-B1 signal transduction.

19. The method of claim 15, wherein the antibody or antigen-binding fragment thereof competitively inhibits a reference monoclonal antibody VX15/2503 or MAb67 from specifically binding to SEMA4D.

20. The method of claim 15, wherein the antibody or antigen-binding fragment thereof comprises a variable heavy chain (VH) comprising VHCDRs 1-3 comprising SEQ ID NOs 6, 7, and 8, respectively, and a variable light chain (VL) comprising VLCDRs 1-3 comprising SEQ ID NOs 14, 15, and 16, respectively.

21. The method of 20, wherein the VH and VL comprise, respectively, SEQ ID NO: 9 and SEQ ID NO: 17 or SEQ ID NO: 10 and SEQ ID NO: 18.

22. The method of claim 15, wherein the at least one huntingtin (HTT)-lowering agent is an antisense oligonucleotide (ASO).

23. The method of claim 22, wherein the ASO is an allele-selective ASO or a non-selective ASO.

24. The method of claim 23, wherein the ASO is a non-selective ASO.

25. The method of claim 15, wherein the ASO and the antibody or antigen-binding fragment thereof are administered separately or concurrently.

26. The method of claim 25, wherein administration of the combination of the isolated antibody or antigen-binding fragment thereof and the HTT-lowering agent results in enhanced therapeutic efficacy relative to administration of the isolated binding molecule or the immune modulating therapy alone.

27. The method of claim 25, wherein administration of the combination of the isolated antibody or antigen-binding fragment thereof and the HTT-lowering agent results in improvement of neuropsychiatric symptoms, cognitive symptoms, motor dysfunction, brain atrophy, metabolic activity, or any combination thereof.

28. The method of claim 27, wherein the improvement of neuropsychiatric symptoms is selected from the group consisting of reduced anxiety-like behavior, improved spatial memory, increased locomotion, and any combination thereof.
